(19) United States
(12) Patent Application Publication
(10) Pub. No.: US 2007/0232647 A1
(43) Pub. Date: Oct. 4, 2007

(54) Use of Ppar Agonists to Treat Ruminants

(76) Inventors: Leopold Franz Goetze, Sandwich (GB); Marcus Eugene Kehrli JR., Ames, IA (US); Anthony Paul Ricketts, Kalamazoo, MI (US); Patrick Carl Taube, Kalamazoo, MI (US)

Correspondence Address: PHARMACIA & UPJOHN, 7000 Portage Road, KZO-300-104, KALAMAZOO, MI, 49001, US

(21) Appl. No.: 11/597,679

(22) PCT Filed: May 13, 2005
(86) PCT No.: PCT/IB05/01501

§ 371 Date: Mar. 15, 2007

Related U.S. Application Data
(60) Provisional application No. 60/574,136, filed on May 25, 2004.

(51) Int. Cl. A61K 31/4709; A23K 1/16; A23K 1/18; A61K 31/19; A61K 31/192; A61P 1/00; A61P 15/00; A61P 37/00; A61P 3/00; A61P 1/16; A61K 31/422; A61K 31/427; A61K 31/451; A61K 31/47

(52) U.S. Cl. 514/310; 514/317; 514/365; 514/374

(57) ABSTRACT

The use of a PPAR agonist in the manufacture of a medicament to increase ruminant serum glucose concentrations, and preferably, the use for the palliative, prophylactic or curative treatment of ruminant disease associated with reduced serum glucose concentration. The ruminant disease associated with reduced serum glucose concentration includes fatty liver syndrome, dystocia, immune dysfunction, impaired immune function, toxification, primary ketosis, secondary ketosis, downer cow syndrome, indigestion, inappetence, retained placenta, displaced abomasum, mastitis, (endo-)-metritis, infertility, low fertility, lameness, subacute rumen acidosis and inadequate nutrient intake associated with stress e.g. heat, poor housing, overcrowding, shipping, dominance or illness, and to increase milk quality and yield.

### FIELD OF THE INVENTION

The invention described herein relates to the novel use of a peroxisome proliferator-activated receptor (PPAR) agonist, to increase ruminant serum glucose levels. In particular, the invention provides the use of a PPAR agonist in the treatment of ruminant diseases associated with reduced serum glucose concentrations. As a separate aspect, there is also provided the use of a PPAR agonist in the treatment of feline hepatic lipidosis.

### BACKGROUND OF THE INVENTION

Peroxisome Proliferator Activated Receptors (PPAR) are implicated in a number of biological processes and disease states including hypercholesterolemia, dyslipidemia, and diabetes. PPARs are members of the nuclear receptor superfamily of transcription factors that includes steroid, thyroid, and vitamin D receptors. They play a role in controlling expression of proteins that regulate lipid and carbohydrate metabolism and are activated by fatty acids and fatty acid metabolites. There are three PPAR subtypes PPAR alpha, PPAR beta (also sometimes referred to as PPAR delta)and PPAR gamma. Each receptor shows a different pattern of tissue expression, and differences in activation by structurally diverse compounds. PPAR receptors are associated with regulation of insulin sensitivity and blood glucose levels, macrophage differentiation, inflammatory response, and cell differentiation. Accordingly, PPARs have been associated with obesity, diabetes, carcinogenesis, inflammation, infertility, hypertension, hyperplasia, atherosclerosis, dyslipidemia, and hypercholesterolemia, (J. Berger, D. E. Moller, Annu. Rev. Med. 2002, 53, 409).

In addition, PPAR alpha agonists lower plasma triglycerides and LDL cholesterol and are therefore useful in treating hypertriglyceridemia, dyslipidemia and obesity. PPAR gamma is associated with the development of non-insulin-dependent diabetes mellitus (NIDDM), hypertension, coronary artery disease, dyslipidemia and certain malignancies. Finally, activation of PPAR beta has been demonstrated to increase HDL levels. (Leibowitz, WO97/28149, August 1997.) More recently, a PPAR beta selective agonist was reported to have shown a dose-related increase in serum HDL-C and decrease in LDL-C and VLDL-TG in insulin-resistant middle aged rhesus monkeys. (W. R. Oliver et al., PNAS, v. 98, pp. 5306-5311, 2001).

All of the PPAR genes have been shown to decrease glucose levels in plasma, (F. A. Gordon, E. Fayard, F. Picard, J. Auwerx, Annual Reviews of Physiology, 2003, 65, 261). The PPAR alpha gene has been implicated in a number of metabolic processes by regulating genes involved in gluconeogenesis, ketogenesis, fatty acid uptake and oxidation in mammals (M. C. Sugden, K. Bulmer, G. F. Gibbons, B. L. Knight, M. J. Holness, Biochem J, 2002, 364, 361). PPAR alpha agonists have been shown to downregulate enzymes in the liver of mice involved in gluconeogenesis (A. Hermanowski-Vosatka, D. Gerhold, S. S. Mundt, V. A. Loving, M. Lu, Y. Chen, A. Elbecht, M. Wu, T. Doebber, L. Kelly, D. Milot, Q. Guo, P. Wang, M. Ippolito, Y. Chao, S. D. Wright, R. Thieringer, Biochemical and Biophysical Research communications, 2000, 279, 330). Free fatty acids affect glucose utilisation, and upon treatment with PPAR alpha agonists, glucose utilisation is improved (B. Jacotot, D. Mathe, J. C. Fruchart, Proceedings of the Xith International Symposium on Atheroschlerosis, Paris, 5-9 Oct. 1997, (1998), 33;). PPAR alpha agonists have also been shown to decrease glucose levels (K. Kuwabara, K. Murakami, M. Todo, T. Aoki, T. Asaki, M. Murai, J. Yano, J. Pharmacol Exp. Ther, 2004). Also, PPAR alpha may be involved in the regulation of insulin secretion, this increases glucose disposal (M. C. Sugden, M. J. Holness, Diabetes, 2004, 53, Suppl. 1, S71). Interestingly, glucose down-regulates the expression of PPAR alpha in the pancreatic beta cell, (R. Roduit, J. Morin, F. Masse, L. Segall, E. Roche, C. B. Newgard, F. Jeannet-Assimacopoulos, M. Prentki, J. Biological Chemistry, 2000, 275, 46, 35799), thus, chronic elevated glucose levels may be involved in reduced fat oxidation and lipid detoxification. Therefore a fine balance needs to be achieved in order to correct a negative energy balance without causing detrimental effects to lipid metabolism.

The PPAR gamma gene is an important regulator of lipid and glucose homeostasis. Improved insulin sensitivity is thought to be due to transcription of genes involved in glucose disposal (Diabetes, 2004, 53, Suppl 1., S60). The PPAR beta gene has also been shown to increase glucose disposal. However, it is unclear whether PPAR genes play a role in fatty acid or carbohydrate processes in ruminants. Also, factors affecting PPAR gene expression and their responsiveness to endogenous ligands in cattle are unknown. Under normal conditions, ruminants rely almost exclusively on gluconeogenesis from propionate in the liver to meet their glucose requirements, and unlike monogastric mammals, little glucose is absorbed directly from the digestive tract.

Dairy cows have been genetically selected for increased milk yield, (which in some cows exceeds 15,000 kg per lactation). In order to maintain this output, significant energy input from feed is required by the cow. Disease, stress, and/or parturition can compromise the cow's appetite often resulting in reduced energy input and overall negative energy balance.

Energy balance is defined as energy intake minus energy output and an animal is described as being in negative energy balance if energy intake is insufficient to meet the demands on maintenance and production (eg milk). A cow in negative energy balance has to find the energy to meet the deficit from its body reserves. Thus cows in negative energy balance tend to lose body condition and liveweight, with cows that are more energy deficient tending to lose condition and weight at a faster rate. It is important that the energy balance and overall health of the cow is managed well in the transition period, since this interval is critically important to the subsequent health, production, and profitability in dairy cows throughout the lactation cycle.

For example, high reproductive efficiency (such as high pregnancy rates per service) has been linked to a satisfactory transition period (J. F. Roche, D. Mackey, M. D. Diskin, Animal Reproduction Science, 2000, 60-61, 703). Also, negative energy balance and disease parameters associated with negative energy balance during the transition period can reduce fertility by increasing interval to first ovulation and inhibiting follicular development (M. C. Lucy, J. Dairy Science, 2001, 84, 1277). The optimal calving interval has been defined in order to time consecutive lactation cycles such that milk production is maximised. Recently, herd fertility has been decreasing in many regions, and as a consequence, time between consecutive lactations is increasing, resulting in a decrease in milk yield over time (or decreased economical efficiency in producing milk).

Milk yield can also be affected directly during the transition period; milk yield lost in early lactation can dramatically affect the yield for the entire lactation, resulting in significant economic loss, (J. K. Drackley, XXII World Buiatrics Congress, Hannover, 2002, 224; J. K. Drackley, J. Dairy science, 1999, 82, 2259).

A particular problem that is still unresolved is the substantial metabolic adjustment required to provide substrates for milk synthesis after calving. Energy deficit in early parturition is prevalent, and although reviews provide some insight into biological events, further understanding in this area is required (C. K. Reynolds, P. C. Aikman, B. Lupoli, D. J. Humphries, D. E. Beever, J. Dairy Science, 2003, 86, 1201; A. W. Bell, J Anim Sci, 1995, 73, 2804; J. K. Drackley, H. M. Dann, N. B. Litherland and J. P Underwood, California Animal Nutrition Conference, Fresno, Calif., USA, May 13-14 2003, 1-16; J. K. Drackley, J. Dairy science, 1999, 82, 2259; R R Grummer, J Anim Sci, 1995, 73, 2820). Lactogenesis (G. C. Waghorn, R. L. Baldwin, J Dairy Sci, 1984, 67, 531) increases the demand for glucose as a precursor for lactose synthesis, and amino acids and fatty acids for milk synthesis from 1 day prepartum, with further demands postpartum. Unfortunately, supplementing glucose in drinking water of transition cows has no effect on serum glucose concentrations or energy balance, and may rather cause ruminal acidosis than be of any benefit (V. R. Osborne, K. E. Leslie, B. W. McBride, Canadian Journal of Animal Science, 2002, 427). Intravenous infusion of glucose into lactating cows had no effect on milk production (D. M. Amaral, J. J. Veenhuizen, J. K. Drackley, M. H. Cooley, A. D. McGilliard, J. W. Young, J. Dairy Sci., 1990, 73, 1244). Furthermore, treatment of cows in established lactation with glucocorticoids decreased milk production despite increasing blood glucose concentrations (R. K. Braun, E. N. Bergman, T. F. Albert, J. A. V. M. A. 1970, 157, 7, 941).

Some work has been performed in sheep showing that responsiveness to insulin decreases during late pregnancy and postpartum, thus increasing lipolysis, NEFA (non esterified fatty acids) mobilisation, amino acid mobilisation and sparing glucose utilisation (Bell and references therein-see above). In sheep at onset of lactation, the lack of lipogenesis, and low utilisation of glucose and acetate by adipose is associated with low plasma levels of insulin. Insulin sensitivity in cattle has not been investigated.

The aim of this invention is to increase endogenous glucose for improvement of energy balance associated with stress, disease and during the transition period.

Feed intake is diminished by stress, disease and during the transition period, thus insufficient propionate is available for gluconeogenesis. Catabolism of amino acids from the diet or from skeletal muscle contributes significantly to glucose synthesis. Long chain fatty acids (or non esterified fatty acids, NEFAs) are also mobilised from body fat. NEFAs, already elevated from around 7 days prepartum, are a significant source of energy to the cow during the early postpartum period, and the greater the energy deficit the higher the concentration of NEFA in the blood. The circulating NEFAs are taken up by the liver and are oxidised to carbon dioxide or ketone bodies, including 3-hydroxybutyrate, by mitochondria, or reconverted via esterification into triglycerides and stored. Furthermore, the capacity of the liver for synthesising very low density lipoproteins to export triglycerides from the liver is limited.

Significantly, if NEFA uptake by the bovine liver becomes excessive, accumulation of ketone bodies can lead to ketosis, and excessive storage of triglycerides may lead to fatty liver. Fatty liver impairs normal liver functions such as gluconeogenesis, and some detoxification processes. The syndrome of fatty liver may result, which can lead to prolonged recovery for other disorders, increased incidence of health problems, and development of “downer cows” that die.

Thus, transition cow sequelae include, fatty liver syndrome, ketosis, low disease resistance, (displaced abomasums, lameness,) immune dysfunction, (mastitis, metritis,) poor reproductive performance (irregular oestrus, prolonged calving interval, poor foetal viability, ovarian cysts, metritis, retained placenta), reduced milk production (peak milk yield, 305 day milk yield). Fatty liver has largely developed by the day after parturition and precedes an induced (secondary) ketosis. It usually results from increased esterification of NEFA absorbed from blood due to negative energy balance coupled with the low ability of ruminant liver to secret triglycerides as very low-density lipoproteins. By improving energy balance, or by treating the negative energy balance, the negative extent of the sequelae will be reduced.

Outside of the transition period, treatment with PPAR agonists will be helpful when the cow is suffering from diarrhea, bacterial infection, poor reproductive performance, displaced abomasum, shock, immunodeficiency, pneumonia, electrolyte imbalance, pain, ketosis, inappetance, reduced feed intake; due to correction of energy balance by elevation of plasma glucose concentration to normal physiological levels.

Response to PPAR alpha agonists is species dependant with some species responding better than others (G. D. Cappon, R. C. M. Liu, S. R. Frame, M. E. Hurtt, Drug and Chemical Toxicology, 2002, 25, 3, 255).

Most recently Drackley has hypothesised that high fat diets prepartum may have increased PPAR alpha expression, resulting in increased hepatic oxidation and decreased esterification of fatty acids in transition cow liver tissue. However, the interplay of biological processes is complicated as described, and knowledge of the important genes, enzymes and endogenous substrates required to optimise the energy balance in cows is limited. Furthermore, it is not known how modification of PPAR expression will effect milk production or quality, lipolysis or gluconeogenesis, since NEFA's are critical substrates for both milk and glucose biosynthesis.

U.S. Provisional Patent Application No. 60/574171, which shares the priority date of the present invention, discloses the use of PPAR agonists as described in International Patent Application Publication Number (WO) 04/048334, in treating negative energy balance in ruminants.

There is a general need for a safe, effective medicament to increase ruminant serum glucose levels. In particular, there is a need for a medicament to treat ruminant disease associated with reduced serum glucose concentrations. There is a particular need for a medicament for ruminants such as sheep and cattle, more particularly for periparturient sheep and cattle, especially for periparturient dairy cows.

There is also a need for a safe, effective treatment of ruminant disease associated with reduced serum glucose concentrations, wherein the disease includes primary and secondary ketosis, downer cow syndrome, indigestion, inappetence, retained placenta, displaced abomasum, impaired immune function, mastitis, (endo-)-metritis, infertility, low fertility, lameness, subacute rumen acidosis and inadequate nutrient intake associated with stress e.g. heat, poor housing, overcrowding, shipping, dominance or illness.

In particular, there is a need for a medicament to both increase ruminant serum glucose levels and treat fatty liver syndrome.

The treatment is preferably administered easily orally or parenterally, preferably does not present residues in meat and/or milk, and preferably does not require a withholding period. It is also preferably non-toxic to feed and animal handlers

We have discovered a novel use of a PPAR agonist to increase ruminant serum glucose concentration. In particular, we have discovered a novel use of a PPAR agonist for the palliative, prophylactic or curative treatment of ruminant disease associated with reduced serum glucose concentration.

Accordingly, one aspect of the invention is the use of a PPAR agonist, in the manufacture of a medicament to increase ruminant serum glucose concentration.

Another aspect of the invention is a method of increasing ruminant serum glucose concentration, which comprises administration to a ruminant of an effective amount of a PPAR agonist.

Further aspects of the invention are as defined in the description and claims.

A preferred use is the use of the group of PPAR agonists described in Annex A, Annex B and Annex C below.

### DESCRIPTION OF THE DRAWINGS

FIG. 1 shows the change in bovine serum glucose concentration after administration of two PPAR agonists, Compound X and Compound Y;

FIG. 2 describes the change in bovine serum glucose concentration after administration of a PPAR agonist, Compound A;

FIG. 3 shows bovine liver triglyceride content after administration of a PPAR agonist, Compound Z;

FIG. 4 shows bovine serum NEFA levels after administration of a PPAR agonist, Compound Z;

FIG. 5 describes ine serum NEFA levels after administration of a PPAR agonist, Compound A;

FIG. 6 describes the average daily milk yield in one hundred twenty four pregnant, non-lactating cows treated by a PPAR agonist, ‘COMPOUND’, against placebo.

### SUMMARY OF THE INVENTION

The present invention provides the use of a PPAR agonist in the manufacture of a medicament to increase ruminant serum glucose concentration.

Another aspect of the invention is the use of a PPAR agonist in the manufacture of a medicament to increase ruminant serum glucose concentrations, with the proviso that a compound of formula I, as disclosed in Annex A, is not used.

A preferred aspect of the invention is the use of a PPAR alpha selective agonist.

An alternative aspect of the invention is the use of a PPAR gamma selective agonist.

A further alternative aspect of the invention is the use of a PPAR beta selective agonist.

Another aspect of the invention is the use of a PPAR agonist more selective for PPAR alpha and gamma than PPAR beta.

An alternative aspect of the invention is the use of a PPAR agonist more selective for PPAR beta and gamma than PPAR alpha.

A further alternative aspect of the invention is the use of a PPAR agonist more selective for PPAR beta and alpha than PPAR gamma.

A preferred aspect of the invention is the use of a PPAR agonist of a formula selected from the formulae disclosed in Annex A, Annex B or Annex C.

An alternative aspect of the invention is the use of a PPAR agonist of a formula selected from the formulae disclosed in Annex B or Annex C.

Another aspect of the invention is the use of a PPAR agonist of formula I disclosed in Annex A.

A preferred aspect of the invention is the use of a PPAR agonist of a formula selected from the formulae disclosed in Annex C.

A more preferred aspect of the invention is the use of a PPAR agonist compound selected from the compounds disclosed in Annex A, Annex B or Annex C.

Even more preferred is the use of a compound selected from the compounds disclosed in Annex C.

An alternative preferred use is when the PPAR agonist is of a formula selected from the formulae disclosed in Annex C, under the heading PCT/IB04/001178, or PCT/IB04/00038, or PCT/IB04/001159. Most preferably, the PPAR agonist is selected from the preferred compounds disclosed in Annex C under the headings PCT/IB04/001178, or PCT/IB04/00038, or PCT/IB04/001159.

A preferred aspect of the invention is the use of a PPAR agonist in the manufacture of a medicament for the palliative, prophylactic or curative treatment of ruminant diseases associated with reduced serum glucose concentrations.

Another aspect of the invention is the use of a PPAR agonist in the manufacture of a medicament to increase ruminant serum glucose concentrations, wherein the excessive accumulation of triglycerides in liver tissue is also prevented or alleviated, and/or the excessive elevation of non-esterified fatty acid levels in serum is also prevented or alleviated.

Another aspect of the invention is the use of a PPAR agonist in the manufacture of a medicament for the palliative, prophylactic or curative treatment of ruminant diseases associated with reduced serum glucose concentrations, wherein the excessive accumulation of triglycerides in liver tissue is also prevented or alleviated, and/or the excessive elevation of non-esterified fatty acid levels in serum is also prevented or alleviated.

Preferably, the ruminant disease associated with reduced serum glucose concentrations is selected from fatty liver syndrome, dystocia, immune dysfunction, toxification, primary ketosis, secondary ketosis, downer cow syndrome, indigestion, inappetence, retained placenta, displaced abomasum, impaired immune function, mastitis, (endo-)-metritis, infertility, low fertility, lameness, subacute rumen acidosis and inadequate nutrient intake associated with stress e.g. heat, poor housing, overcrowding, shipping, dominance or illness.

More preferably, the ruminant disease is selected from fatty liver syndrome, primary ketosis, downer cow syndrome, (endo-)-metritis and low fertility.

Preferably, the PPAR agonist is administered to a ruminant with reduced, or lower than typical, serum glucose concentrations.

Yet another aspect of the invention is the use of a PPAR agonist for the manufacture of a medicament for the treatment of negative energy balance in ruminants, preferably for the treatment of diseases associated with negative energy balance in ruminants.

A further aspect of the invention is the use of a PPAR agonist for the manufacture of a medicament for the treatment of fatty liver syndrome and/or diseases associated with fatty liver syndrome.

Preferably, the diseases associated with negative energy balance in ruminants, or associated with fatty liver syndrome, are selected from fatty liver syndrome, dystocia, immune dysfunction, impaired immune function, toxification, primary ketosis, secondary ketosis, downer cow syndrome, indigestion, inappetence, retained placenta, displaced abomasum, mastitis, (endo-)-metritis, infertility, low fertility, fasciolosis and lameness.

More preferably, where the invention is the use of a PPAR agonist for the manufacture of a medicament for the treatment of negative energy balance, or of disease associated with negative energy balance in ruminants, or for the treatment of fatty liver syndrome and/or diseases associated with fatty liver syndrome, the PPAR agonist is of a formula selected from the formulae disclosed in Annex C, under the heading PCT/IB04/001178, or PCT/IB04/00038, or PCT/IB04/001159. Most preferably, the PPAR agonist is selected from the preferred compounds disclosed in Annex C under the headings PCT/IB04/001178, or PCT/IB04/00038, or PCT/IB04/001159.

Another aspect of the invention is the use of a PPAR agonist in the manufacture of a medicament to increase ruminant serum glucose concentrations, and for the improvement of fertility, including decreased return to service rates, normal oestrus cycling, improved conception rates, and improved foetal viability.

Yet another aspect of the invention is the use of a PPAR agonist in the manufacture of a medicament for the management of effective homeorhesis to accommodate parturition and lactogenesis.

Another aspect of the invention is the use of a compound of formula 1, in the manufacture of a medicament to increase ruminant serum glucose concentrations and for improving or maintaining the functioning of the ruminant liver and homeostatic signals during the transition period.

In one aspect of the invention, the PPAR agonist is administered during the period from 30 days prepartum to 70 days postpartum.

In another aspect of the invention, the PPAR agonist is administered prepartum and, optionally, also at parturition.

In yet another aspect of the invention, the PPAR agonist is administered postpartum.

In yet another aspect of the invention, the PPAR agonist is administered at parturition.

More preferably, the PPAR agonist is administered during the period from 3 weeks prepartum to 3 weeks postpartum.

In another aspect of the invention, the PPAR agonist is administered up to three times during the first seven days postpartum.

Preferably, the PPAR agonist is administered once during the first 24 hours postpartum.

In another aspect of the invention, the PPAR agonist is administered prepartum and up to four times postpartum.

In another aspect of the invention, the PPAR agonist is administered at parturition and then up to four times postpartum.

Another aspect of the invention is the use of a PPAR agonist in the manufacture of a medicament to increase ruminant serum glucose concentrations, and to increase ruminant milk quality and/or milk yield.

In a preferred aspect of the invention, the milk quality increase is seen in a reduction in the levels of ketone bodies in ruminant milk.

In another aspect of the invention, peak milk yield is increased.

Preferably, the ruminant is a cow or sheep.

In another aspect of the invention, an overall increase in ruminant milk yield is obtained during the 305 days of the bovine lactation period.

In another aspect of the invention, an overall increase in ruminant milk yield is obtained during the first 60 days of the bovine lactation period.

Preferably, the overall increase in ruminant milk yield, or the increase in peak milk yield, or the increase in milk quality, is obtained from a dairy cow.

In one aspect of the invention, the increase in ruminant milk quality and/or milk yield is obtained after administration of a PPAR agonist to a healthy ruminant.

In another aspect of the invention, there is provided a PPAR agonist for increasing ruminant serum glucose concentration.

In a preferred aspect of the invention, there is provided a PPAR agonist for the palliative, prophylactic or curative treatment of ruminant disease associated with reduced blood glucose concentration, wherein, preferably, the disease is selected from fatty liver syndrome, dystocia, immune dysfunction, impaired immune function, toxification, primary and secondary ketosis, downer cow syndrome, indigestion, inappetence, retained placenta, displaced abomasum, mastitis, (endo-)-metritis, infertility, low fertility and lameness.

In another aspect of the invention, there is provided a PPAR agonist for increasing ruminant serum glucose concentrations, and for increasing ruminant milk quantity and/or quality.

In another aspect of the invention, there is provided a kit for increasing ruminant serum glucose concentration, comprising:

a) a PPAR agonist, and

b) optionally, one or more pharmaceutically acceptable carriers, excipients or diluents, and

c) packaging for containing a) and optionally b)

Preferably, the kit is for the palliative, prophylactic or curative treatment of ruminant disease associated with reduced blood glucose concentration.

More preferably, the kit is for the palliative, prophylactic or curative treatment of fatty liver syndrome, dystocia, immune dysfunction, impaired immune function, toxification, primary and secondary ketosis, downer cow syndrome, indigestion, inappetence, retained placenta, displaced abomasum, mastitis, (endo-)-metritis, infertility, low fertility and lameness.

Even more preferably, the kit further comprises instructions for increasing ruminant serum glucose concentration or for the palliative, prophylactic or curative treatment of ruminant disease associated with reduced blood glucose concentration.

As a separate aspect, there is provided the use of a PPAR agonist, particularly a PPAR alpha agonist, in the treatment of feline hepatic lipidosis. Particular PPAR agonists for use in this invention are selected from those compounds presented below, including those highlighted as being preferred, and in the subject matter of the patents and patent applications which are recited below or incorporated by reference. Feline hepatic lipidosis is characterized by an accumulation of lipids in the liver, leading to a fatty liver and an impairment of hepatic functions resulting in jaundice, vomiting, anorexia, inappetence, and lethargy. Hepatic lipidosis has been defined as the accumulation of triglyceride in the liver >5% of the gross liver weight. In FHL, hepatic accumulation of triglyceride may occur with increased uptake of nonesterified fatty acids (NEFA), impaired fatty acid oxidation, disturbances in VLDL assembly and secretion or disruption in any combination of the above pathways. References include: Center S. A. Feline hepatic lipidosis. Vet Clin North Am Small Anim Pract. January 2005;35(1):225-69. Review; Blanchard G. et al Plasma lipids, lipoprotein composition and profile during induction and treatment of hepatic lipidosis in cats and the metabolic effect of one daily meal in healthy cats. J Anim Physiol Anim Nutr (Berl). April 2004;88(3-4):73-87; Pazak H. E. Characterization of serum lipoprotein profiles of healthy, adult cats and idiopathic feline hepatic lipidosis patients. J Nutr. December 1998;128(12 Suppl):2747S-2750S; and Blanchard G. Dietary L-carnitine supplementation in obese cats alters carnitine metabolism and decreases ketosis during fasting and induced hepatic lipidosis. J Nutr. February 2002;132(2):204-10.

The “transition period” means from 30 days prepartum to 70 days postpartum

The term “treating”, “treat”, “treats” or “treatment” as used herein includes prophylactic, palliative and curative treatment.

The term “cow” as used herein includes heifer, primiparous and multiparous cow.

The term “PPAR alpha agonist” means an agonist more selective for PPAR alpha than gamma or beta.

The term “PPAR gamma agonist” means an agonist more selective for PPAR gamma than alpha or beta.

The term “PPAR beta agonist” means an agonist more selective for PPAR beta than alpha or gamma.

A “PPAR beta agonist” is also known as a “PPAR delta agonist”. Where used herein, these definitions are to be considered interchangeable.

Where “PPAR beta” or “beta” or “PPAR beta agonist” is used herein, it is taken to mean also “PPAR delta” or “delta” or “PPAR delta agonist”, respectively. A dual or mixed PPAR agonist may be defined as a pharmacological active compound, that at use rate (therapeutic dose) produces levels high enough to effect more than one class of receptors at the receptor site.

“Negative energy balance” as used herein means that energy via food does not meet the requirements of maintenance and production (milk).

“Healthy ruminant” means where the ruminant does not show signs of the following indications: fatty liver syndrome, dystocia, immune dysfunction, impaired immune function, toxification, primary and secondary ketosis, downer cow syndrome, indigestion, inappetence, retained placenta, displaced abomasum, mastitis, (endo-)-metritis, infertility, low fertility and/or lameness.

Milk “quality” as used herein refers to the levels in milk of protein, fat, lactose, somatic cells, and ketone bodies. An increase in milk quality is obtained on an increase in fat, protein or lactose content, or a decrease in somatic cell levels or ketone bodies levels.

An increase in milk yield can mean an increase in milk solids or milk fat or milk protein content, as well as, or instead of, an increase in the volume of milk produced.

“Excessive accumulation of triglycerides” as used herein means greater than the physiological triglyceride content of 10% w/w in liver tissue.

“Excessive elevation of non-esterified fatty acid levels in serum” as used herein means non-esterified fatty acid levels of greater than 800 μmol/L in serum.

Unless otherwise specified, “prepartum” means 3 weeks before calving until the day of calving.

Unless otherwise specified, “postpartum” means from when the newborn is “expelled” from the uterus to 6 weeks after the newborn was expelled from the uterus.

“At parturition” means the 24 hours after the newborn was expelled from the uterus.

“Periparturient” or “periparturient period” means the “transition period”.

By “pharmaceutically acceptable” is meant the carrier, diluent, vehicle, excipient, and/or salt must be compatible with the other ingredients of the formulation, and not deleterious to the recipient thereof.

As used herein, “therapeutically effective amount of a compound” means an amount that is effective to exhibit therapeutic or biological activity at the site(s) of activity in a ruminant, without undue adverse side effects (such as undue toxicity, irritation or allergic response), commensurate with a reasonable benefit/risk ratio when used in the manner of the present invention.

The mention of use of compounds in the present invention, shall at all times be understood to include all active forms of such compounds, including, for example, the free form thereof, e.g., the free acid or base form, and also, all prodrugs, polymorphs, hydrates, solvates, tautomers, stereoisomers, e.g., diastereomers and enantiomers, and the like, and all pharmaceutically acceptable salts as described above, unless specifically stated otherwise. It will also be appreciated that the use of suitable active metabolites of such compounds, in any suitable form, are also included herein.

PPAR FRET Assay

Measurement of coactivator recruitment by a nuclear receptor after receptor-ligand association is a method for evaluating the ability of a ligand to produce a functional response through a nuclear receptor. The PPAR FRET (Fluorescence Resonance Energy Transfer) assay measures the ligand-dependent interaction between nuclear receptor and coactivator. GST/PPAR (α,β, and γ) ligand binding domain (LBD) is labeled with a europium-tagged anti-GST antibody, while an SRC-1 (Sterol Receptor Coactivator-1) synthetic peptide containing an amino terminus long chain biotin molecule is labeled with streptavidin-linked allophycocyanin (APC). Binding of ligand to the PPAR LBD causes a conformational change that allows SRC-1 to bind. Upon SRC-1 binding, the donor FRET molecule (europium) comes in close proximity to the acceptor molecule (APC), resulting in fluorescence energy transfer between donor (337 nm excitation and 620 nm emission) and acceptor (620 nm excitation and 665 nm emission). Increases in the ratio of 665 nm emission to 620 nm emission is a measure of the ability of the ligand-PPAR LBD to recruit SRC-1 synthetic peptide and therefore a measure of the ability of a ligand to produce a functional response through the PPAR receptor.

[1] GST/PPAR LBD Expression. The human PPARα LBD (amino acids 235-507) is fused to the carboxy terminus of glutathione S-transferase (GST) in pGEX-6P-1 (Pharmacia, Piscataway, N.J.). The GST/PPARα LBD fusion protein is expressed in BL21[DE3]pLysS cells using a 50 uM IPTG induction at room temperature for 16 hr (cells induced at an A₆₀₀of ˜0.6). Fusion protein is purified on glutathione sepharose 4B beads, eluted in 10 mM reduced glutathione, and dialyzed against 1× PBS at 4° C. Fusion protein is quantitated by Bradford assay (M. M. Bradford, Analst. Biochem. 72:248-254; 1976), and stored at ˜20° C. in 1× PBS containing 40% glycerol and 5 mM DTT.

[2] FRET Assay. The FRET assay reaction mix consists of 1× FRET buffer (50 mM Tris-Cl pH 8.0, 50 mM KCl, 0.1 mg/ml BSA, 1 mM EDTA, and 2 mM DTT) containing 20 nM GST/PPARα LBD, 40 nM of SRC-1 peptide (amino acids 676-700, 5′-long chain biotin-CPSSHSSLTERHKILHRLLQEGSPS-NH₂, purchased from American Peptide Co., Sunnyvale, Calif.), 2 nM of europium-conjugated anti-GST antibody (Wallac, Gaithersburg, Md.), 40 nM of streptavidin-conjugated APC (Wallac), and control and test compounds. The final volume is brought to 100 ul with water and transferred to a black 96-well plate (Microfuor B, Dynex (Chantilly, Va.)). The reaction mixes are incubated for 1 hr at 4° C. and fluorescence is read in Victor 2 plate reader (Wallac). Data is presented as a ratio of the emission at 665 nm to the emission at 615 nm.

Selectivity Measurements

Transient transfections assay using the HepG2 hepatoma cell line.

HepG2 cells were transiently transfected with an expression plasmids encoding hPPARα, hPPAβ or mPPARγ chimeric receptors and a reporter containing the yeast upstream activating sequence (UAS) upstream of the viral E1B promoter controlling a luciferase reporter gene. In addition, the plasmid pRSVβ-gal was used to control for transfection efficiency. HepG2 cells were grown in DMEM supplemented with 10% FBS and 1 μM non-essential amino acid. On the first day, cells were split into 100 mm dishes at 2.5×10⁶/dish and incubated overnight at 37° C./5% CO₂. On the second day the cells were transiently transfected with plasmid DNA encoding a chimeric receptor, the luciferase reporter gene; and β-gal. For each 100 mm dish, 15 μg of lucifease reporter (PG5E1b) DNA, 15 μg of Gal4-PPAR chimeric receptor DNA, and 1.5 μg of β-gal plasmid DNA were mixed with 1.4 ml of opti-MEM in the tube. 28 μl of LipoFectamine-2000 reagent was added to 1.4 ml of opti-MEM in the tube, and incubate for 5 min at RT. The diluted Lipofectamine-2000 reagent was combined with the DNA mixture, and incubate for 20 min at RT. After fresh medium was added to each 100 mm dish of cells, 2.8 ml of Lipofectamine2000-DNA mixture was added dropwise to the 100 mm dish containing 14 ml of medium, and incubate 37° C. overnight. On day three cells were trypsinized off the 100 mm dishes and re-plated on 96 well plates. Cells were plated at 2.5×10⁴ cells per well in 150 μl of media and 50 μl of compound diluted by media was added. The concentrations of reference agents and test compound added were in the range from 50 μM to 50 pM. After addition of compounds, the plates were incubated at 37° C. for 24 hours. Subsequently cells were washed once with 100 μl of PBS, lysed, and processed for measuring luciferase and β-gal activity using Dual-Light luciferase kit from Tropix®, according to the manufacturer's recommendations, on an EG&G Bethold MicroLumat LB96P luminometer. Hep G2-hBeta EC₅₀ values (“EC₅₀β”) and Hep G2-hAlpha EC_{50.} values, (“EC₅₀α”) were obtained using the GraphPad Prism™ program. EC₅₀ is the concentration at which the PPAR mediated transcriptional response reaches one-half of its maximal response.

Serum Glucose Levels:

### EXAMPLE 1

Twelve mid-lactation cows were selected to investigate the pharmacokinetic and pharmacodynamic properties of 2 PPAR alpha agonists, compound “X” and compound “Y”. Both compounds were administered by IV and SC routes, as outlined in the table below.

| Treatment | Dose | No. Cows |
|---|---|---|
| T1 compound “X” | 0.5 mg/kg IV | 3 |
| T2 compound “X” | 0.5 mg/kg SC | 3 |
| T3 compound “Y” | 0.5 mg/kg IV | 3 |
| T4 compound “Y” | 0.5 mg/kg SC | 3 |

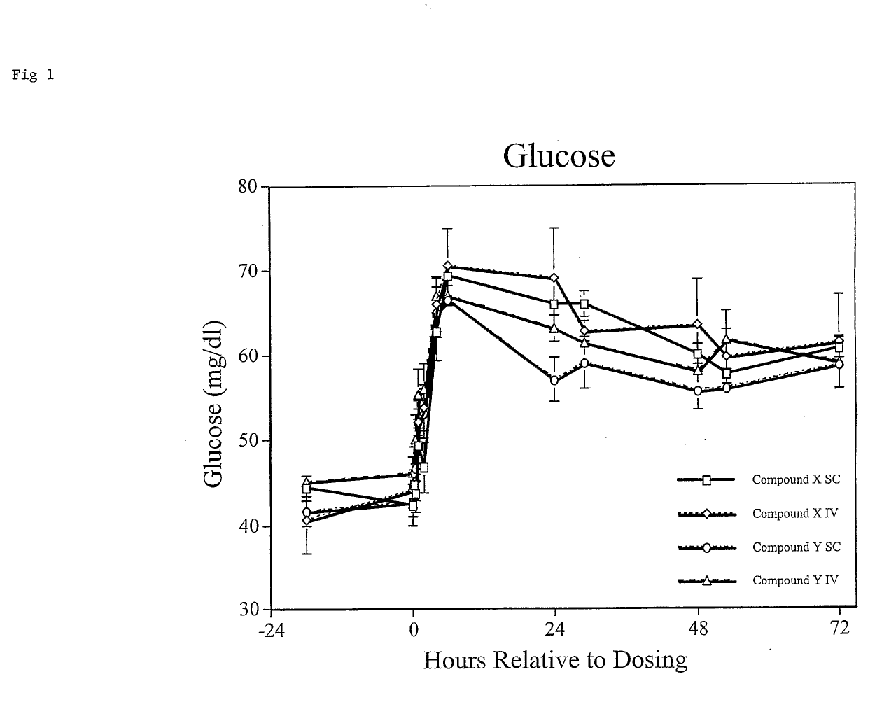

All animals were bled one day prior, and 15 minutes prior, to compound administration. Blood samples were collected from animals treated via IV at 5 and 10 minutes post compound administration. Blood samples were collected from all animals at 0.5, 1, 2, 4, 6, 24, 30, 48, 54, and 72 hours post compound administration. Samples were analyzed for glucose concentrations using a Dade Behring Dimension RXL serum chemistry analyzer. Treatment by both compounds, regardless of route of administration, caused an increase in glucose, relative to baseline values (see FIG. 1). Other commercially available, standard apparatus for testing glucose levels can also be used.

Compound X is 2-Methyl-5-(4′-methyl-biphenyl-4-ylsulfamoyl)-benzoic acid, having the structure below and compound Y is 3-[3-(1-Carboxy-1-methyl-ethoxy)-phenyl]-piperidine-1-carboxylic acid, 4-isopropyl-benzyl ester, having the structure below:

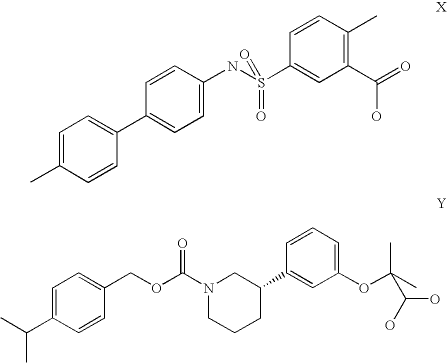

### EXAMPLE 2

Ten mid-lactation cows, fed a low starch/high fat diet were selected to investigate the effect of administration upon circulating levels of glucose. Animals had been fed the low starch/high fat diet for one week prior to administration, and remained on the same diet throughout the study.

| Treatment | Dose | | No. Cows |
|---|---|---|---|
| T1 Saline | 3.0 | mL SC | 5 |
| T2 Compound A | 0.5 | mg/kg SC | 5 |

All animals were bled 5 minutes prior to compound administration. Blood samples were collected from all animals at 2, 4, 6, 8, 24, 32, and 48 hours post-compound administration. In this Example, Compound A is the PPAR alpha agonist (3S)-3-[3-(1-Carboxy-1-methyl-ethoxy)-phenyl]-piperidine-1-carboxylic acid 4-trifluoromethyl-benzyl ester.

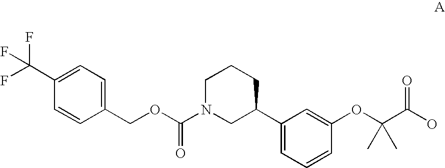

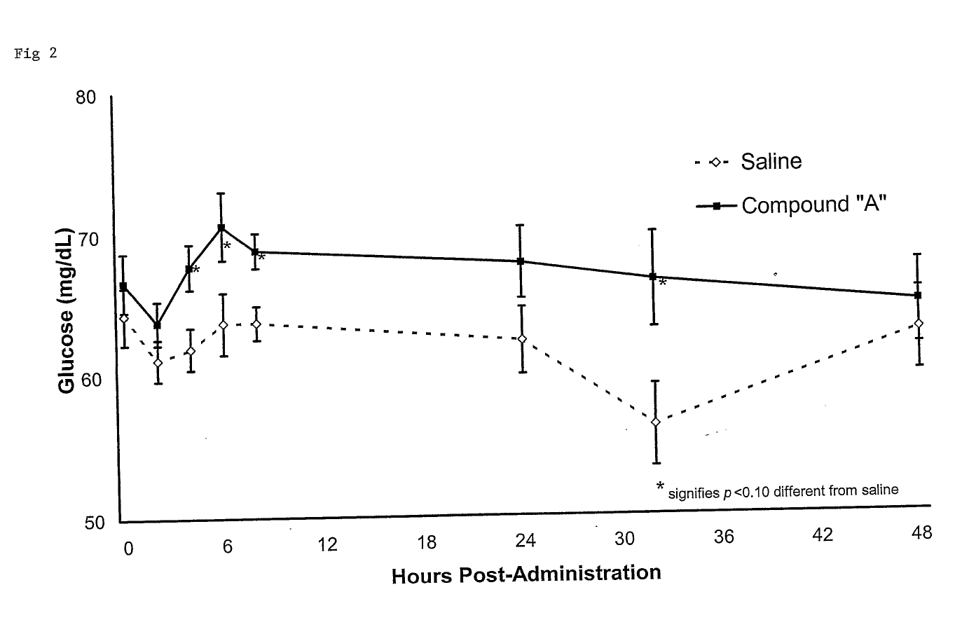

Samples were analyzed for glucose concentrations using an Olympus AU640 analyzer. Treatment by the compound caused a transient increase in glucose, relative to saline controls. Results are shown in FIG. 2.

Determination of Changes in Blood Non-Esterified Fatty Acid (NEFA) Concentrations and Liver Triglycerides Levels:

### EXAMPLE 1

Compounds were administered once or several times in the transition period at dose levels predicted to be effective by comparing results of in-vitro receptor affinity tests in laboratory species and pharmacokinetic evaluations in cattle. NEFA levels were determined via standard laboratory methods, for example, using the commercial WAKO NEFA kit (Wako Chemical Co., USA, Dallas, Tex., 994-75409), and liver triglyceride content was determined using the method as described in the literature (J. K. Drackley, J. J. Veenhuizen, M. J. Richard and J. W. Young, J Dairy Sci, 1991, 74, 4254)).

All animals were obtained from a commercial dairy farm approximately thirty days prior to anticipated calving date. The cows were moved into separate building, approximately 10-14 days prior to their anticipated calving dates and switched to the TMR-Close-Up dry diet. Enrollment of animals in the study began approximately 7 days prior to their anticipated calving dates. The animals were moved to the “on-test” pen, weighed and were locked each AM into feed stanchions. At that time, appropriate doses were administered and appropriate blood samples obtained (see table below).

| | | | | | Post |
|---|---|---|---|---|---|
| | | | Pre Partum Dosing | Treat- | Partum |
| | Dose | Animals | (every other day = | ment | Dosing |
| | (mg/ | per | eod − beginning | at | (eod 4 |
| Treatment | kg) | Treatment | targeted day − 7) | Calving | doses) |
| T01 | — | 9 | X | X | |
| Vehicle | | | | | |
| Control | | | | | |
| T02 | 0.5 | 8 | X | X | |
| Compound Z | | | | | |
| T03 | 0.5 | 11 | | X | X |
| Compound Z | | | | | |
| T04 | 0.5 | 9 | | X | |
| Compound Z | | | | | |

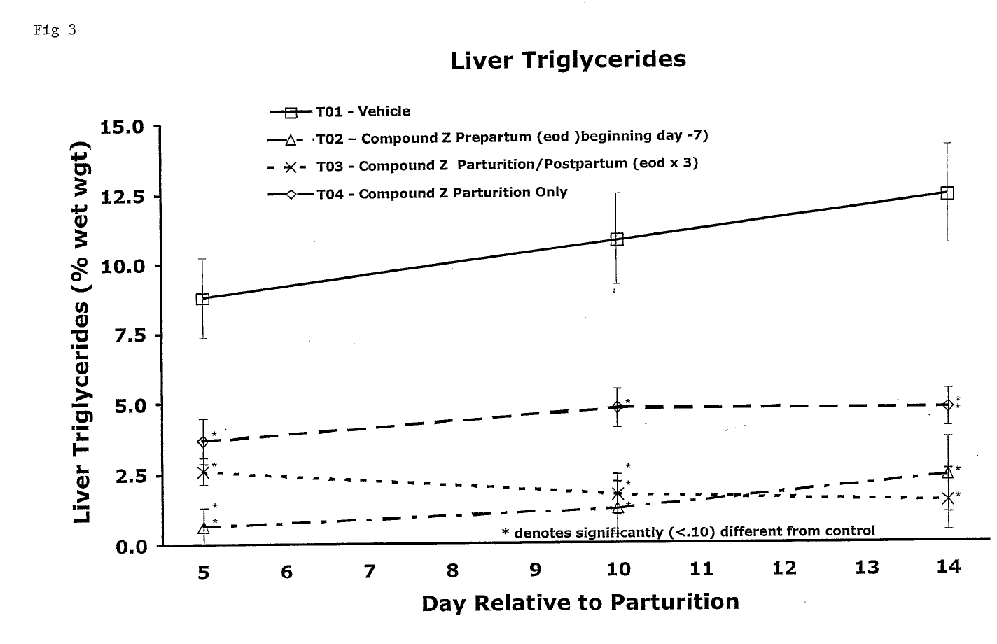

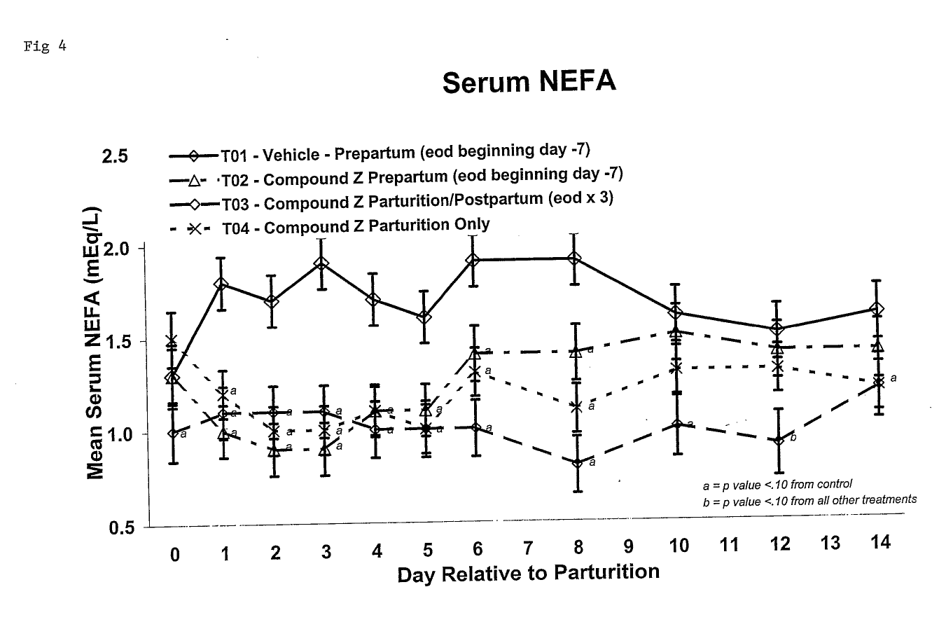

As soon as possible post-calving (˜30 minutes) the cow was transferred to the freestall barn for the next scheduled milking (6:00 hrs and 19:00 hrs). Treatments on postpartum animals were administered every other day through day 8. Pre and post-calving NEFA samples were analyzed using the WAKO NEFA-C test kit (#994-75409). Post-calving liver biopsies were performed on all cows on days 5, 10 and 14 post-calving. Tissues were transported on ice and stored frozen at −70° F. At the conclusion of the study, samples were analysed of liver triglyceride levels using the method described by Drackley, J. K. et al. (1991, J Dairy Sci (74):4254-4264). All animals treated with compound “Z”, (3S)-3-[3-(1-Carboxy-1-methyl-ethoxy)-phenyl]-piperidine-1-carboxylic acid, 4-trifluoromethyl-benzyl ester, exhibited significantly lower serum NEFA levels from Day 1 (after calving) until the end of the study as compared to controls, with the exceptions of T02 on day 8. All treatment regimens significantly lowered liver triglyceride levels compared to placebo at all time points measured (Days 5, 10 and 14 postcalving) (See FIGS. 3 and 4).

### EXAMPLE 2

Eleven early-lactation cows, fed a low starch/high fat diet were selected to investigate the effect of administration of Compound “A” upon circulating levels of serum NEFA. Animals had been fed the low starch/high fat diet for one week prior to administration, and remained on the same diet throughout the study.

| Treatment | Dose | | No. Cows |
|---|---|---|---|
| T1 Saline | 3.0 | mL SC | 6 |
| T2 Compound “A” | 0.5 | mg/kg SC | 5 |

All animals were bled 5 minutes prior to compound administration. Blood samples were collected from all animals at 11, 24, 35, 48, 59, 72, 83, 96, 107, 120, 131, and 144 hours post-compound administration. In this Example, Compound A is the PPAR alph/beta agonist {5-Methoxy-2-methyl-4-[4-(4-trifluoromethyl-benzyloxy)-benzylsulfanyl]-phenoxy}-acetic acid, having the structure shown below.

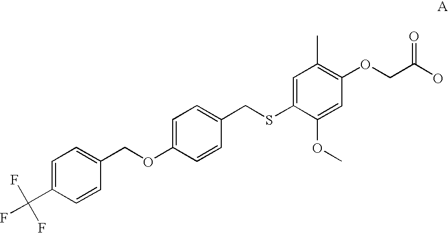

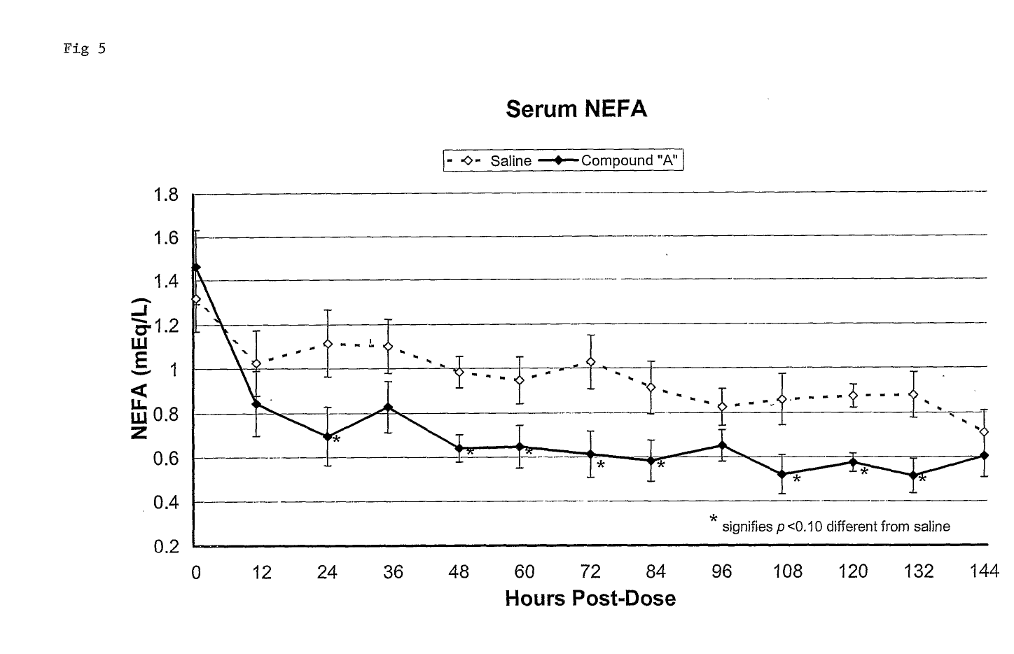

Samples were analyzed for NEFA using WAKO NEFA-C Kit. Treatment by the compound caused an decrease in circulating serum NEFA values relative to pre-treatment values, and relative to saline controls. Results are shown in FIG. 5.

Ketone Bodies

Levels of ketone bodies in serum can be measured by standard methods well known to the person skilled in the art, for example, by using the commercially available kits for this purpose, including Sigma BHBA kit of order number 310-A.

Levels of NEFAs, triglycerides and ketone bodies considered ‘higher than normal’ or ‘excessive’ are:

NEFA's >800 μmol/L in serum.

Triglycerides >10% w/w in liver tissue.

Ketone bodies >1.2 μmol/L in serum.

Normal values for serum glucose levels in adult cattle 3.3-3.9-4.4 mmol/l (range and mean level), which is equivalent to 59-70-79 mg/dl or mg/100 ml. “Reduced”, or “lower than typical” serum glucose levels, means lower than the normal values, for the ruminant.

Milk Content:

Machines to assay for milk protein, fat, or lactose content are commercially available (MilkoScan™ 50, MilkoScan™ 4000, MilkoScan™ FT 6000 available from Foss Group).

Machines to assay for somatic cell content are also commercially available (Fossomatic™ FC, Fossomatic™ Minor available from Foss Group).

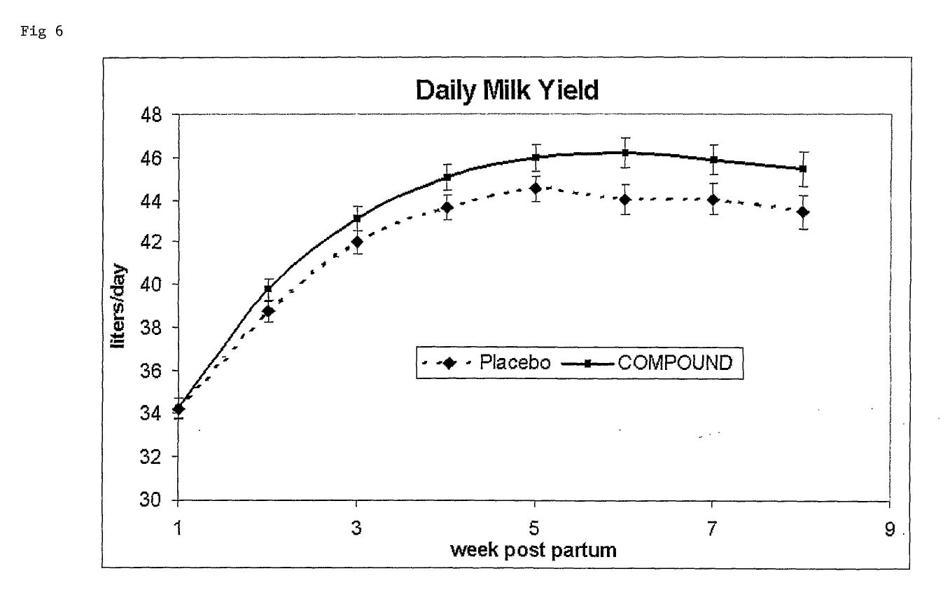

One hundred twenty four pregnant, non-lactating Holstein cows were allocated to two treatment groups (placebo and COMPOUND (at approximately 0.5 mg/kg). In this Example COMPOUND is (3S)-3-[3-(1-Carboxy-1-methyl-ethoxy)-phenyl]-piperidine-1-carboxylic acid, 4-trifluoromethyl-benzyl ester. Animals were allowed to calve, treated by subcutaneous injection on the day of calving and on day five post-calving. Disease events and daily milk production were recorded for the following sixty days. The average daily milk yield in the treated cows was increased from 41.8 to 43.2 kg/day (p=0.052). Results are shown in FIG. 6.

Compounds used in this invention may be administered alone or in combination with one or more other compounds of the invention or in combination with one or more other drugs (or as any combination thereof).

For example, compounds of this invention can also be mixed with one or more biologically active compounds or agents selected from sedatives, analgesics, antiinflammatories, analeptics, antibacterials, antidiarrhoeals, anti-endotoxin, antifungals, respiratory stimulants, corticosteroids, diuretics, parasiticides, electrolyte preparations and nutritional supplements, growth promoters, hormones, and metabolic disease treatments, giving an even broader spectrum of veterinary or agricultural utility.

Examples of suitable active compounds or agents are found below:

Rumen Amylase and or glucosidae inhibitors, e.g. acarbose

Sedative: alpha adrenergic agonists, e.g. xylazine,

Analgesics and antiinflammatories: Lignocaine, Procaine, flunixin, oxytetracycline, ketoprofen, meloxicam and carprofen.

Analeptics: Etamiphylline, Doxapram, Diprenorphine, Hyoscine, Ketoprofen, Meloxicam, Pethidine, Xylazine and Butorphanol,

Antibacterials: Chlortetracycline, Tylosin, Amoxycillin, Ampicillin, Aproamycin, Cefquinome, Cephalexin, Clavulanic acid, Florfenicol, Danofloxacin, Enrofloxacin, Marbofloxacin, Framycetin, Procaine penicillin, procaine benzylpenicillin, Benzathine penicillin, sulfadoxine, Trimethoprim, sulphadimidine, baquiloprim, streptomycin, dihydrostreptomycin, sulphamethoxypyridazine, sulphamethoxypuridazine, oxytetracycline, flunixin, tilmicosin, cloxacillin, ethyromycin, neomycin, nafcillin, Aureomycin, lineomycin, cefoperazone, cephalonium, oxytetracycline, formosulphathiazole, sulphadiazine and zinc.

Antidiarrhoeals: Hyoscine, Dipyrone, charcoal, attapulgite, kaolin, Isphaghula husk,

Anti-endotoxins: Flunixin, ketoprofen,

Antifungals: Enilconazole, Natamycin,

Respiratory stimulants: florfenicol,

Corticosteroids: dexamethasone, betamethasone,

Diuretics: frusemide,

Parasiticides—amitraz, deltamethrin, moxidectin, doramectin, alpha cypermethrin, fenvalerate, eprinomectin, permethrin, ivermectin, abamectin, ricobendazole, levamisole, febantel, triclabendazole, fenbendazole, albendazole, netobimin, oxfenazole, oxyclozanide, nitroxynil, morantel,

Electrolyte preparations and nutritional supplements: dextrose, lactose, propylene glycol, whey, glucose, glycine, calcium, cobalt, copper, iodine, iron, magnesium, manganese, phosphorous, selenium, zinc, Biotin, vitamin B₁₂, Vitamin E, and other vitamins,

Growth Promoters: monensin, flavophospholipol, bambermycin, salinomycin, tylosin,

Hormones: chorionic gonadotrophin, serum gonadotrophin, atropine, melatonin, oxytocin, dinoprost, cloprostenol, etiproston, luprostiol, buserelin, oestradiol, progesterone, and bovine somatotropin.

Metabolic Disease Treatments: calcium gluconate, calcium borogluconate, propylene glycol, magnesium sulphate,

Compounds of this invention can also be mixed with one or more biologically active compounds or agents selected from antiprotozoals such as imidocarb, bloat remedies such as dimethicone and poloxalene, and probiotics such as Lactobacilli and streptococcus.

Other compounds which may be mixed with compounds for use in the invention include rumen protected choline; DCAD; amino acids e.g. glutamine, lysine, serine, methionine, alanine, aspartamine; probiotics e.g. Propionibacterium, Teichomycin A2; yeasts; glucocorticoids: glucose precursors e.g. glucagon, propylene glycol, propionic acid, propyl esters, propyl alcohol, lactose, glycerol, pyruvate; vegetable oils, e.g. safflower; fish oils; unsaturated fatty acids e.g CLA; algae extracts (to increase omega fatty acids); plant sterols e.g. ergosterol; alpha-ketoisocaproate; vitamin D; calcium and magnesium salts; miscellaneous branded treatments: Reassure, Rally, MEGALAC, Fermenten, Rumensin crc bolus; and miscellaneous antiinflammatory agents: prednisolone; antibiotic ionophores e.g. nigericin, tetronasin; antibiotics: cefamezin and metronidazole.

As a preferred feature of the present invention, alpha amylase and alpha glucosidase inhibitors e.g. acarbose, may be combined with PPAR agonists, preferably PPAR alpha agonists, for use according to the present invention.

Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

With respect to their use in ruminants, the compounds may be administered alone or in a formulation appropriate to the specific use envisaged.

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Other possible ingredients include anti-oxidants, colourants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001).

The compounds of the invention may also be administered directly into the blood stream, into muscle, or into an internal organ. Suitable means for parenteral administration include bolus, intravenous, intraarterial, intraperitoneal, intrathecal, intraventricular, intraurethral, intrasternal, intracranial, intramuscular and subcutaneous. Ear implants can also be used. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of the PPAR agonist(s) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

The compounds of the invention may also be administered topically to the skin or mucosa, that is, dermally or transdermally. Typical formulations for this purpose include drenches, gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999). Pour-on or spot-on formulations may be prepared by dissolving the active ingredient in an acceptable liquid carrier vehicle such as butyl digol, liquid paraffin or a non-volatile ester, optionally with the addition of a volatile component such as propan-2-ol. Alternatively, pour-on, spot-on or spray formulations can be prepared by encapsulation, to leave a residue of active agent on the surface of the animal. Injectable formulations may be prepared in the form of a sterile solution which may contain other substances, for example enough salts or glucose to make the solution isotonic with blood.

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as I-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 to 1000 μg of the compound of formula (I). The overall daily dose will typically be in the range 100 μg to 100 mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate. Silicone rubber based intravaginal devices can be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and-non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Acceptable liquid carriers include vegetable oils such as sesame oil, glycerides such as triacetin, esters such as benzyl benzoate, isopropyl myristate and fatty acid derivatives of propylene glycol, as well as organic solvents such as pyrrolidin-2-one and glycerol formal. The formulations are prepared by dissolving or suspending the active ingredient in the liquid carrier such that the final formulation contains from 0.01 to 10% by weight of the active ingredient.

Such formulations are prepared in a conventional manner in accordance with standard veterinary practice.

These formulations will vary with regard to the weight of active compound contained therein, depending on the species of host animal to be treated, the severity and type of infection and the body weight of the host. For parenteral, topical and oral administration, typical dose ranges of the active ingredient are 0.05 to 5 mg per kg of body weight of the animal. Preferably the range is 0.01 to 1 mg per kg.

As an alternative the compounds may be administered to a ruiminant with the drinking water or feedstuff and for this purpose a concentrated feed additive or premix may be prepared for mixing with the normal animal feed or drink.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a PPAR agonist in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to ruminants, the total daily dose of the compounds of the invention is typically in the range 0.05 mg/kg to 5mg/kg depending, of course, on the mode of administration. For example, oral administration may require a total daily dose of from 0.05 mg/kg to 5 mg/kg, while an intravenous dose may only require from 0.01 mg/kg to 1 mg/kg. The total daily dose may be administered in single or divided doses. The veterinarian will readily be able to determine doses for individual ruminants according to age, weight and need.

### FORMULATION EXAMPLES

In the formulations which follow, “active ingredient” means a compound used in the present invention.

Formulation 1: Solution for parenteral administration
Solution of active ingredient will be prepared as follows:

| Ingredient | Quantity (mg/5 ml) | |
|---|---|---|
| Active ingredient | 1-750 | |
| Potassium hydroxide | 0.1-75 | |
| Sodium dihydrogen phosphate | 0-50 | |
| Disodium hydrogen phosphate | 0-100 | |
| Methyl Paraben | 0-40 | |
| Water | Up to 5 | ml |

Or

Formulation 1a: Solution for parenteral administration
Solution of active ingredient will be prepared as follows:

| Ingredient | Quantity (mg/5 ml) | |
|---|---|---|
| Active ingredient | 1-750 | |
| Potassium hydroxide | 0-75 | |
| Sodium hydroxide | 0-75 | |
| Sodium dihydrogen phosphate | 0-50 | |
| Disodium hydrogen phosphate | 0-100 | |
| PVP | 0-50 | |
| Methyl Paraben | 0-40 | |
| Water | Up to 5 | ml |

Formulation 2: Solution for parenteral administration
Solution of active ingredient will be prepared as follows:

| Ingredient | Quantity (mg/5 ml) | |
|---|---|---|
| Active ingredient | 1-750 | |
| Sodium dihydrogen phosphate | 0-50 | |
| Disodium hydrogen phosphate | 0-100 | |
| Methyl Paraben | 0-40 | |
| Water | Up to 5 | ml |

Or

Formulation 3: Solution for parenteral administration
Solution of active ingredient will be prepared as follows:

| Ingredient | Quantity (mg/5 ml) | |
|---|---|---|
| Active ingredient | 1-500 | |
| Hydroxy propyl β-cyclodextrin | 10-4000 | |
| Methyl Paraben | 0-40 | |
| Water | Up to 5 | ml |

Formulation 4: Solution for subcutaneous administration
Solution of active ingredient will be prepared as follows:

| Ingredient | Quantity (mg) |
|---|---|
| Active ingredient | 1-500 |
| Glycerol Formal | 100-10000 |

Formulation 5: Gelatin Capsules
Hard gelatin capsules are prepared using the following:

| Ingredient | Quantity (mg/capsule) |
|---|---|
| Active ingredient | 1-500 |
| Starch, NF | 0-1000 |
| Starch flowable powder | 0-250 |
| Silicone fluid 350 centistokes | 0-45 |

Formulation 6: Tablets - A tablet formulation is prepared
using the ingredients below:

| Ingredient | Quantity (mg/tablet) |
|---|---|
| Active ingredient | 0.25-500 |
| Cellulose, microcrystalline | 100-1000 |
| Silicon dioxide, fumed | 10-1000 |
| Stearate acid | 5-50 |

The components are blended and compressed to form tablets.

Alternatively, tablets each containing 1-500 mg of active ingredients are made up as follows:

Formulation 7: Tablets

| Ingredient | Quantity (mg/tablet) |
|---|---|
| Active ingredient | 1-500 |
| Starch | 45-200 |
| Cellulose, microcrystalline | 35-100 |
| Polyvinylpyrrolidone (as 10% solution in water) | 4-20 |
| Sodium carboxymethyl cellulose | 4.5 |
| Magnesium stearate | 0.5-2 |
| Talc | 1-5 |

The active ingredients, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50°-60° C. and passed through a No. 18 mesh U.S. sieve. The sodium carboxymethyl starch, magnesium stearate, and talc, previously passed through a No. 60 U.S. sieve, are then added to the granules which, after mixing, are compressed on a tablet machine to yield tablets.

Suspensions each containing 1-750 mg of active ingredient per 5 ml dose are made as follows:

Formulation 4: Suspensions

| Ingredient | Quantity (mg/5 ml) | |
|---|---|---|
| Active ingredient | 1-750 | mg |
| Sodium carboxymethyl cellulose | 50 | mg |
| Syrup | 1.25 | mg |
| Benzoic acid solution | 0.10 | mL |
| Flavor | q.v. | |
| Color | q.v. | |
| Purified Water to | 5 | mL |

The active ingredient is passed through a No. 45 mesh U.S. sieve and mixed with the sodium carboxymethyl cellulose and syrup to form a smooth paste. The benzoic acid solution, flavor, and color are diluted with some of the water and added, with stirring. Sufficient water is then added to produce the required volume.

### ANNEX A

This annex refers to the subject matter of U.S. Provisional Patent Application No. 60/429506, filed on 26 Nov. 2003 and published with International Patent Application Publication Number WO04/048334, published on 10 Jun. 2004. The subject matter of U.S. 60/429506 was recited in full in the priority application for the present application. For brevity, according to U.S. 60/429506 and WO04/048334, the compounds of formula (I), the compounds described by the preferred sub-formulae, the exemplified compounds of the listed preferred compounds are all incorporated by reference into the present application and are part of the present invention. For the avoidance of doubt, the following examples from 60/429506 are compounds suitable for use according to the present invention:

Example 1 2—: (3-{1-[(4-Isopropyl-phenyl)-acetyl]-piperidin-3-yl}-phenoxy)-2-methyl-propionic acid benzyl ester

Example 1-12—: (3-{1-[(3-Methoxy-phenyl)-acetyl]-piperidin-3-yl}-phenoxy)-2-methyl -propionic acid

Example 1-22—: (3-{1-[(4-Methoxy-phenyl)-acetyl]-piperidin-3-yl}-phenoxy)-2-methyl-propionic acid

Example 1-32: (3-{1-[(4-Fluoro-phenyl)-acetyl]-piperidin-3-yl}-phenoxy)-2-methyl-propionic acid

Example 1-4: 2-(3-{1-[(4-Hydroxy-phenyl)-acetyl]-piperidin-3-yl}-phenoxy)-2-methyl-propionic acid

Example 1-5: 2-{3-[1-(4-Isopropyl-benzoyl)-piperidin-3-yl]-phenoxy}-2-methyl-propionic acid

Example 1-6: 2-(3-{1-[(2,4-Dimethoxy-phenyl)-acetyl]-piperidin-3-yl}-phenoxy)-2-methyl-propionic acid

Example 1-7: 2-Methyl-2-(3-{1-[(4-trifluoromethyl-phenyl)-acetyl]-piperidin-3-yl}-phenoxy)-propionic acid

Example 1-8: 2-(3-{1-[3-(3-Methoxy-phenyl)-propionyl]-piperidin-3-yl}-phenoxy)-2-methyl-propionic acid

Example 1-9: 2-Methyl-2-{3-[1-(pyridin-2-yl-acetyl)-piperidin-3-yl]-phenoxy}-propionic acid

Example 1-10: 2-Methyl-2-{3-[1-(pyridin-3-yl-acetyl)-piperidin-3-yl]-phenoxy}-propionic acid

Example 1-11: 2-Methyl-2-{3-[1-(pyridin-4-yl-acetyl)-piperidin-3-yl]-phenoxy}-propionic acid

Example 1-12: 2-[3-(1-Cyclohexylacetyl-piperidin-3-yl)-phenoxy]-2-methyl-propionic acid

Example 1-13: (S)-2-(3-{1-[(4-Isopropyl-phenyl)-acetyl]-piperidin-3-yl}-phenoxy)-2-methyl-propionic acid

Example 1-14: (R)-2-(3-{1-[(4-Isopropyl-phenyl)-acetyl]-piperidin-3-yl}-phenoxy)-2-methyl-propionic acid

Example 1-15: 2-[3-(1-Isobutyryl-piperidin-3-yl)-phenoxy]-2-methyl-propionic acid

Example 1-16: 2-Methyl-2-[3-(1-phenylacetyl-piperidin-3-yl)-phenoxy]-propionic acid

Example 1-17: 2-Methyl-2-{3-[1-(3-phenyl-propionyl)-piperidin-3-yl]-phenoxy}-propionic acid

Example 1-18: 2-Methyl-2-[3-(1-m-tolylacetyl-piperidin-3-yl)-phenoxy]-propionic acid

Example 1-19: 2-Methyl-2-{3-[1-(pyridine-2-carbonyl)-piperidin-3-yl]-phenoxy}-propionic acid

Example 1-20: 2-Methyl-2-{3-[1-(pyridine-3-carbonyl)-piperidin-3-yl]-phenoxy}-propionic acid

Example 1-21: 2-[3-(1-Benzoyl-piperidin-3-yl)-phenoxy]-2-methyl-propionic acid

Example 1-22: 2-(3-{1-[(3-Fluoro-phenyl)-acetyl]-piperidin-3-yl}-phenoxy)-2-methyl-propionic acid

Example 1-23: 2-(3-{1-[(3-Chloro-phenyl)-acetyl]-piperidin-3-yl}-phenoxy)-2-methyl-propionic acid

Example 1-24: 2-(3-{1-[(4-Chloro-phenyl)-acetyl]-piperidin-3-yl}-phenoxy)-2-methyl-propionic acid

Example 1-25: 2-Methyl-2-(3-{1-[(4-trifluoromethoxy-phenyl)-acetyl]-piperidin-3-yl}-phenoxy)-propionic acid

Example 1-26: 2-Methyl-2-{3-[1-(3-piperidin-1-yl-propionyl)-piperidin-3-yl]-phenoxy}-propionic acid

Example 1-27: 2-Methyl-2-{3-[1-(3-methyl-butyryl)-piperidin-3-yl]-phenoxy}-propionic acid

Example 1-28: 2-(3-{1-[(4-Ethoxy-phenyl)-acetyl]-piperidin-3-yl}-phenoxy)-2-methyl-propionic acid

Example 1-29: 2-(3-{1-[(2-Methoxy-phenyl)-acetyl]-piperidin-3-yl}-phenoxy)-2-methyl-propionic acid

Example 1-30: 2-Methyl-2-[3-(1-o-tolylacetyl-piperidin-3-yl)-phenoxy]-propionic acid

Example 1-31: 2-Methyl-2-[3-(1-p-tolylacetyl-piperidin-3-yl)-phenoxy]-propionic acid

Example 1-32: 2-(3-{1-[(3,5-Dimethoxy-phenyl)-acetyl]-piperidin-3-yl}-phenoxy)-2-methyl-propionic acid

Example 1-33: 2-Methyl-2-(3-{1-[(3-trifluoromethyl-phenyl)-acetyl]-piperidin-3-yl}-phenoxy)-propionic acid

Example 1-34: 2-(3-{1-[(3,5-Bis-trifluoromethyl-phenyl)-acetyl]-piperidin-3-yl}-phenoxy)-2-methyl-propionic acid

Example 1-35: 2-Methyl-2-(3-{1-[(3-trifluoromethoxy-phenyl)-acetyl]-piperidin-3-yl}-phenoxy)-propionic acid

Example 1-36: 2-Methyl-2-(3-{1-[3-(3-trifluoromethoxy-phenyl)-propionyl]-piperidin-3-yl}-phenoxy-propionic acid

Example 1-37: 2-Methyl-2-{3-[1-(piperidin-1-yl-acetyl)-piperidin-3-yl]-phenoxy}-propionic acid

Example 1-38: 2-Methyl-2-{3-[1-(morpholin-4-yl-acetyl)-piperidin-3-yl]-phenoxy}-propionic acid

Example 1-39: 2-Methyl-2-{3-[1-(piperazin-1-yl-acetyl)-piperidin-3-yl]-phenoxy}-propionic acid

Example 1-40: 2-(3-{1-[(1H-Benzoimidazol-2-yl)-acetyl]-piperidin-3-yl}-phenoxy)-2-methyl-propionic acid

Example 1-41: 2-{3-[1-(Benzo[1,3]dioxol-5-yl-acetyl)-piperidin-3-yl]-phenoxy}-2-methyl-propionic acid

Example 1-42: 2-(3-{1-[(2-Hydroxy-phenyl)-acetyl)-piperidin-3-yl}-phenoxy)-2-methyl-propionic acid

Example 1-43: 2-(3-{1-[(4-tert-Butyl-phenyl)-acetyl]-piperidin-3-yl}-phenoxy)-2-methyl-propionic acid

Example 1-44: 2-(3-{1-[(4-Ethyl-phenyl)-acetyl]-piperidin-3-yl}-phenoxy)-2-methyl-propionic acid

Example 1-45: 2-{3-[1-(4-Isobutyl-benzoyl)-piperidin-3-yl]-phenoxy}-2-methyl-propionic acid

Example 1-46: 2-(3-{1-[(4-Isobutyl-phenyl)-acetyl]-piperidin-3-yl}-phenoxy)-2-methyl-propionic acid

Example 1-47: 2-Methyl-2-(3-{1-[4-(2,2,2-trifluoro-1-hydroxy-1-trifluoromethyl-ethyl)-benzoyl]-piperidin-3-yl}-phenoxy)-propionic acid

Example 1-48: (S)-2-(3-{1-[(4-tert-Butyl-phenyl)-acetyl]-piperidin-3-yl}-phenoxy)-2-methyl-propionic acid

Example 1-49: (S)-2-Methyl-2-(3-{1-[(4-trifluoromethoxy-phenyl)-acetyl]-piperidin-3-yl}-phenoxy)-propionic acid

Example 1-50: (R)-2-Methyl-2-(3-{1-[(4-trifluoromethoxy-phenyl)-acetyl]-piperidin-3-yl}-phenoxy)-propionic acid

Example 1-51: (R)-2-(3-{1-[(4-tert-Butyl-phenyl)-acetyl]-piperidin-3-yl}-phenoxy)-2-methyl-propionic acid

Example 1-52: (S)-2-(3-{1-[(4-Cyclohexyl-phenyl)-acetyl]-piperidin-3-yl}-phenoxy)-2-methyl-propionic acid

Example 1-53: (S)-2-(3-{1-[(4-Methanesulfonyl-phenyl)-acetyl]-piperidin-3-yl}-phenoxy)-2-methyl-propionic acid

Example 1-54: (S)-2-{3-[1-(Biphenyl-4-yl-acetyl)-piperidin-3-yl]-phenoxy}-2-methyl-propionic acid

Example 1-55: (S)-2-Methyl-2-{3-[1-(naphthalen-2-yl-acetyl)-piperidin-3-yl]-phenoxy}-propionic acid

Example 1-56: (S)-2-Methyl-2-(3-{1-[4-methyl-2-(4-trifluoromethyl-phenyl)-thiazole-5-carbonyl]-piperidin-3-yl}-phenoxy)-propionic acid

Example 1-57: (S)-2-Methyl-2-{3-[1-(naphthalen-1-yl-acetyl)-piperidin-3-yl]-phenoxy}-propionic acid

Example 1-58: (S)-2-Methyl-2-(3-{1-[(4-trifluoromethyl-phenyl)-acetyl]-piperidin-3-yl}-phenoxy)-propionic acid

Example 1-59: 2-(4-{1-[(4-Isopropyl-phenyl)-acetyl]-piperidin-3-yl}-phenoxy)-2-methyl-propionic acid

Example 1-60: 2-Methyl-2-(4-{1-[(4-trifluoromethyl-phenyl)-acetyl]-piperidin-3-yl}-phenoxy)-propionic acid

Example 1-61: 2-{4-[1-(4-Isopropyl-benzoyl)-piperidin-3-yl]-phenoxy}-2-methyl-propionic acid

Example 1-62: 2-Methyl-2-{4-[1-(pyridin-2-yl-acetyl)-piperidin-3-yl]-phenoxy}-propionic acid

Example 1-63: 2-(4-{1-[3-(4-isopropyl-phenyl)-propionyl]-piperidin-3-yl}-phenoxy)-2-methyl-propionic acid

Example 1-64: (3-{1-[(4-Isopropyl-phenyl)-acetyl]-piperidin-3-yl}-phenoxy)-acetic acid

Example 2: 2-(3-{1-[(4-Isopropyl-phenoxy)-acetyl]-piperidin-3-yl}-phenoxy)-2-methyl-propionic acid

Example 2-1: 2-(3-{1-[2-(4-Isopropyl-phenoxy)-2-methyl-propionyl]-piperidin-3-yl}-phenoxy)-2-methyl-propionic acid

Example 2-2: 2-Methyl-2-(3-{1-[(4-trifluoromethoxy-phenoxy)-acetyl]-piperidin-3-yl}-phenoxy)-propionic acid

Example 2-3: (S)-2-(3-{1-[(4-Isopropyl-phenoxy)-acetyl]-piperidin-3-yl}-phenoxy)-2-methyl-propionic acid

Example 2-4: (R)-2-(3-{1-[(4-Isopropyl-phenoxy)-acetyl]-piperidin-3-yl}-phenoxy)-2-methyl-propionic acid

Example 2-5: (S)-2-Methyl-2-(3-{1-[(4-trifluoromethoxy-phenoxy)-acetyl]-piperidin-3-yl}-phenoxy)-propionic acid

Example 2-6: (R)-2-Methyl-2-(3-{1-[(4-trifluoromethoxy-phenoxy)-acetyl]-piperidin-3-yl}-phenoxy)-propionic acid

Example 2-7: 2-(3-{1-[(3-Isopropyl-phenoxy)-acetyl]-piperidin-3-yl}-phenoxy)-2-methyl-propionic acid

Example 2-8: 2-(3-{1-[(4-tert-Butyl-phenoxy)-acetyl]-piperidin-3-yl}-phenoxy)-2-methyl-propionic acid

Example 2-9: 2-Methyl-2-[3-(1-m-tolyloxyacetyl-piperidin-3-yl)-phenoxy]-propionic acid

Example 2-10: 2-Methyl-2-(3-{1-[(3-trifluoromethyl-phenoxy)-acetyl]-piperidin-3-yl}-phenoxy)-propionic acid

Example 2-11: (S)-2-(3-{1-[(3-Isopropyl-phenoxy)-acetyl]-piperidin-3-yl}-phenoxy)-2-methyl-propionic acid

Example 3: 2-(3-{1-[3-(4-Isopropyl-phenyl)-propionyl]-piperidin-3-yl}-phenoxy)-2-methyl-propionic acid

Example 3-1: 2-Methyl-2-(3-{1-[3-(4-trifluoromethyl-phenyl)-propionyl]-piperidin-3-yl}-phenoxy)-propionic acid

Example 3-2: 2-Methyl-2-(3-{1-[3-(4-trifluoromethoxy-phenyl)-propionyl]-piperidin-3-yl}-phenoxy)-propionic acid

Example 4: 3-[3-(1-Carboxy-1-methyl-ethoxy)-phenyl]-piperidine-1-carboxylic acid 4-isopropyl-phenyl ester

Example 4-1: 3-[3-(1-Carboxy-1-methyl-ethoxy)-phenyl]-piperidine-1-carboxylic acid 3-isopropyl-phenyl ester

Example 4-2: 3-[3-(1-Carboxy-1-methyl-ethoxy)-phenyl]-piperidine-1-carboxylic acid 4-tert-butyl-phenyl ester

Example 4-3: (R)-3-[3-(1-Carboxy-1-methyl-ethoxy)-phenyl]-piperidine-1-carboxylic acid 4-isopropyl-phenyl ester

Example 4-4: (S)-3-[3-(1-Carboxy-1-methyl-ethoxy)-phenyl]-piperidine-1-carboxylic acid 4-isopropyl-phenyl ester

Example 5: 3-[3-(1-carboxy-1-methyl-ethoxy)-phenyl]-piperidine-1-carboxylic acid 4-isopropyl-benzyl ester

Example 5-1: 3-[3-(1-Carboxy-1-methyl-ethoxy)-phenyl]-piperidine-1-carboxylic acid 4-trifluoromethyl-benzyl ester

Example 5-2: (R)-3-[3-(1-Carboxy-1-methyl-ethoxy)-phenyl]-piperidine-1-carboxylic acid 4-isopropyl-benzyl ester

Example 5-3: (S)-3-[3-(1-Carboxy-1-methyl-ethoxy)-phenyl]-piperidine-1-carboxylic acid 4-isopropyl-benzyl ester

Example 5-4: (S)-3-[3-(1-Carboxy-1-methyl-ethoxy)-phenyl]-piperidine-1-carboxylic acid 4-isopropyl-benzyl ester

Example 5-5: (S)-3-[3-(1-Carboxy-1-methyl-ethoxy)-phenyl]-piperidine-1-carboxylic acid 4-ethyl-benzyl ester

Example 5-6: (S)-3-[3-(1-Carboxy-1-methyl-ethoxy)-phenyl]-piperidine-1-carboxylic acid 3-trifluoromethyl-benzyl ester

Example 5-7: (S)-3-[3-(1-Carboxy-1-methyl-ethoxy)-phenyl]-piperidine-1-carboxylic acid 4-trifluoromethoxy-benzyl ester

Example 5-8: (S)-3-[3-(1-Carboxy-1-methyl-ethoxy)-phenyl]-piperidine-1-carboxylic acid benzyl ester

Example 5-9: (S)-3-[3-(1-Carboxy-1-methyl-ethoxy)-phenyl]-piperidine-1-carboxylic acid 4-fluoro-benzyl ester

Example 5-10: (S)-3-[3-(1-Carboxy-1-methyl-ethoxy)-phenyl]-piperidine-1-carboxylic acid 4-fluoro-3-trifluoromethyl-benzyl ester

Example 5-11: (S)-3-[3-(1-Carboxy-1-methyl-ethoxy)-phenyl]-piperidine-1-carboxylic acid 3-fluoro-4-trifluoromethyl-benzyl ester

Example 5-12: (S)-3-[3-(1-Carboxy-1-methyl-ethoxy)-phenyl]-piperidine-1-carboxylic acid 3-trifluoromethoxy-benzyl ester

Example 6: 3-[3-(1-carboxy-1-methyl-ethoxy)-phenyl]-piperidine-1-carboxylic acid 4-isopropyl-benzyl ester

Example 6-1: (3S)-3-[3-(1-Carboxy-1-methyl-ethoxy)-phenyl]-piperidine-1-carboxylic acid 4-trifluoromethyl-benzyl ester

Example 6-2: 3-[3-(1-Carboxy-1-methyl-ethoxy)-phenyl]-piperidine-1-carboxylic acid 4-cyclopropyl-benzyl ester

Example 7: (S)-3-[3-(1-carboxy-1-methyl-ethoxy)-phenyl]-piperidine-1-carboxylic acid methyl ester

Example 7-1: (S)-3-[3-(1-Carboxy-1-methyl-ethoxy)-phenyl]-piperidine-1-carboxylic acid 2-methoxy-ethyl ester

Example 7-2: (S)-3-[3-(1-Carboxy-1-methyl-ethoxy)-phenyl]-piperidine-1-carboxylic acid isopropyl ester

Example 7-3: (S)-3-[3-(1-Carboxy-1-methyl-ethoxy)-phenyl]-piperidine-1-carboxylic acid ethyl ester

Example 7-4: (S)-3-[3-(1-Carboxy-1-methyl-ethoxy)-phenyl]-piperidine-1-carboxylic acid isobutyl ester

Example 7-5: (S)-3-[3-(1-Carboxy-1-methyl-ethoxy)-phenyl]-piperidine-1-carboxylic acid cyclohexylmethyl ester

Example 8: 2-methyl-2-{3-[1-(4-trifluoromethyl-benzylcarbamoyl)-piperidin-3-yl]-phenoxy}-propionic acid

Example 8-1: 2-{3-[1-(4-Isopropyl-benzylcarbamoyl)-piperidin-3-yl]-phenoxy}-2-methyl-propionic acid

Example 8-2: 2-Methyl-2-{3-[1-(4-trifluoromethoxy-benzylcarbamoyl)-piperidin-3-yl]-phenoxy}-propionic acid

Example 8-3: (S)-2-Methyl-2-{3-[1-(4-trifluoromethoxy-benzylcarbamoyl)-piperidin-3-yl]-phenoxy}-propionic acid

Example 8-4: (S)-2-{3-[1-(4-Isopropyl-benzylcarbamoyl)-piperidin-3-yl]-phenoxy}-2-methyl-propionic acid

Example 8-5: (S)-2-{3-[1-(Cyclohexylmethyl-carbamoyl)-piperidin-3-yl]-phenoxy}-2-methyl-propionic acid

Example 8-6: 2-{3-[1-(4-Isopropyl-phenylcarbamoyl)-piperidin-3-yl]-phenoxy}-2-methyl-propionic acid

Example 9: (R)-3-(3-carboxy-4-methyl-phenyl)-piperidine-1-carboxylic acid 4-trifluoromethyl-benzyl ester

Example 9-1: (R)-2-methyl-5-{1-[4-methyl-2-(4-trifluoromethyl-phenyl)-thiazole-5-carbonyl]-piperidin-3-yl}-benzoic acid

Example 9-2: (S)-2-methyl-5-(1-[4-methyl-2-(4-trifluoromethyl-phenyl)-thiazole-5-carbonyl]-piperidin-3-yl}-benzoic acid

Example 9-3: 2-methyl-5-{1-[4-methyl-2-(4-trifluoromethyl-phenyl)-thiazole-5-carbonyl]-piperidin-3-yl}-benzoic acid

Example 9-4: (S)-3-(3-carboxy-4-methyl-phenyl)-piperidine-1-carboxylic acid 4-trifluoromethyl-benzyl ester

Example 9-5: 3-(3-carboxy-4-methyl-phenyl)-piperidine-1-carboxylic acid 4-trifluoromethyl-benzyl ester

Example 9-6: 2-Methyl-5-{1-[(4-trifluoromethoxy-phenyl)-acetyl]-piperidin-3-yl}-benzoic acid

Example 9-7: 5-{1-[(4-Isopropyl-phenyl)-acetyl]-piperidin-3-yl}-2-methyl-benzoic acid

Example 9-8: 2-Methyl-5-{1-[(4-trifluoromethyl-phenyl)-acetyl]-piperidin-3-yl}-benzoic acid

Example 9-9: 2-Methyl-5-{1-[3-(4-trifluoromethyl-phenyl)-acryloyl]-piperidin-3-yl}-benzoic acid

Example 9-10: 5-{1-[3-(4-Isopropyl-phenyl)-acryloyl]-piperidin-3-yl}-2-methyl-benzoic acid

Example 9-11: 2-Methyl-5-{1-[3-(4-trifluoromethyl-phenyl)-propionyl]-piperidin-3-yl}-benzoic acid

Example 9-12: 5-{1-[3-(4-Isopropyl-phenyl)-propionyl]-piperidin-3-yl}-2-methyl-benzoic acid

Example 9-13: 3-(3-Carboxy-4-methyl-phenyl)-piperidine-1-carboxylic acid 4-isopropyl-benzyl ester

Example 9-14: (R)-2-Methyl-5-[1-(4-trifluoromethyl-benzylcarbamoyl)-piperidin-3-yl]-benzoic acid

Example 9-15: (S)-2-Methyl-5-[1-(4-trifluoromethyl-benzylcarbamoyl)-piperidin-3-yl]-benzoic acid

Example 9-16: (R)-3-(3-Carboxy-4-methyl-phenyl)-piperidine-1-carboxylic acid 2-(4-trifluoromethyl-phenyl)-ethyl ester

Example 9-17: 2-Methyl-4-[1-(4-trifluoromethyl-benzoyl)-piperidin-3-yl]-benzoic acid

Example 9-18: 2-Methyl-4-{1-[(4-trifluoromethyl-phenyl)-acetyl]-piperidin-3-yl}-benzoic acid

Example 9-19: 2-Methyl-4-{1-[3-(4-trifluoromethyl-phenyl)-acryloyl]-piperidin-3-yl}-benzoic acid

Example 9-20: 2-Methyl-4-{1-[4-methyl-2-(4-trifluoromethyl-phenyl)-thiazole-5-carbonyl]-piperidin-3-yl}-benzoic acid

Example 9-21: 3-(4-Carboxy-3-methyl-phenyl)-piperidine-1-carboxylic acid 4-trifluoromethyl-benzyl ester

Example 9-22: 4-[1-(4-Isopropyl-benzoyl)-piperidin-3-yl]-2-methyl-benzoic acid

Example 9-23: 4-{1-[(4-Isopropyl-phenyl)-acetyl]-piperidin-3-yl}-2-methyl-benzoic acid

Example 9-24: 4-{1-[3-(4-Isopropyl-phenyl)-acryloyl]-piperidin-3-yl}-2-methyl-benzoic acid

Example 9-25: 3-(4-Carboxy-3-methyl-phenyl)-piperidine-1-carboxylic acid 4-isopropyl-benzyl ester

Example 9-26: 2-Methyl-4-{1-[3-(4-trifluoromethyl-phenyl)-propionyl]-piperidin-3-yl}-benzoic acid

Example 9-27: 4-{1-[3-(4-Isopropyl-phenyl)-propionyl]-piperidin-3-yl}-2-methyl-benzoic acid

Example 9-28: Isomer of 2-methoxy-5-{1-[4-methyl-2-(4-trifluoromethyl-phenyl)-thiazole-5-carbonyl]-piperidin-3-yl}-benzoic acid from L tartaric acid.

Example 9-29: Isomer of 2-methoxy-5-{1-[4-methyl-2-(4-trifluoromethyl-phenyl)-thiazole-5-carbonyl]-piperidin-3-yl}-benzoic acid from D tartaric acid

Example 9-30: 2-Fluoro-5-{1-[4-methyl-2-(4-trifluoromethyl-phenyl)-thiazole-5-carbonyl]-piperidin-3-yl}-benzoic acid

Example 9-3: 1 3-(3-Carboxy-4-fluoro-phenyl)-piperidine-1-carboxylic acid 4-trifluoromethyl-benzyl ester

Example 10: {3-[4-methyl-3-(1H-tetrazol-5-yl)-phenyl]-piperidin-1-yl}-[4-methyl-2-(4-trifluoromethyl-phenyl)-thiazol-5-yl]-methanone

Example 11: (S)-2-Methyl-2-(2-methyl-5-{1-[4-methyl-2-(4-trifluoromethyl-phenyl)-thiazole-5-

Example 11-1: (S)-3-[3-(1-Carboxy-1-methyl-ethoxy)-4-methyl-phenyl]-piperidine-1-carboxylic acid 4-trifluoromethyl-benzyl ester

Example 11-2: (R)-2-Methyl-2-(2-methyl-5-{1-[4-methyl-2-(4-trifluoromethyl-phenyl)-thiazole-5-carbonyl]-piperidin-3-yl}-phenoxy)-propionic acid

Example 11-3: (R)-3-[3-(1-Carboxy-1-methyl-ethoxy)-4-methyl-phenyl]-piperidine-1-carboxylic acid 4-trifluoromethyl-benzyl ester

Examples 11-4, 11-5 and 11-6 were prepared using methods analogous to those described in Example 11 and 11-1.

Example 11-4: 2-Methyl-2-(2-methyl-4-{1-[4-methyl-2-(4-trifluoromethyl-phenyl)-thiazole-5-carbonyl}-piperidin-3-yl}-phenoxy)-propionic acid

Example 11-5: 3-[4-(1-Carboxy-1-methyl-ethoxy)-3-methyl-phenyl]-piperidine-1-carboxylic acid 4-trifluoromethyl-benzyl ester

Example 11-6: (S)-3-[3-(1-Carboxy-1-methyl-ethoxy)-4-methyl-phenyl]-piperidine-1-carboxylic acid 2-(4-trifluoromethyl-phenyl)-ethyl ester and (R)-3-[3-(1-Carboxy-1-methyl-ethoxy)-4-methyl-phenyl]-piperidine-1-carboxylic acid 2-(4-trifluoromethyl-phenyl)-ethyl ester

Example 12: (S)-(2-methyl-5-{1-[4-methyl-2-(4-trifluoromethyl-phenyl)-thiazole-5-carbonyl]-piperidin-3-yl}-phenoxy)-acetic acid.

Example 12-2: (R)-(2-Methyl-5-{1-[4-methyl-2-(4-trifluoromethyl-phenyl)-thiazole-5-carbonyl]-piperidin-3-yl}-phenoxy)-acetic acid

Example 12-3: (R)-3-(3-Carboxymethoxy-4-methyl-phenyl)-piperidine-1-carboxylic acid 4-trifluoromethyl-benzyl ester.

Example 12-4: (2-Methyl-4-{1-[4-methyl-2-(4-trifluoromethyl-phenyl)-thiazole-5-carbonyl]-piperidin-3-yl}-phenoxy)-acetic acid

Example 12-5: 3-(4-Carboxymethoxy-3-methyl-phenyl)-piperidine-1-carboxylic acid 4-

Example 13 C: C,C-Trifluoro-N-(2-methyl-5-{1-[4-methyl-2-(4-trifluoromethyl-phenyl)-thiazole-5-carbonyl]-piperidin-3-yl}-phenyl)-methanesulfonamide

Example 13-13-: [3-(Carboxymethyl-amino)-4-methyl-phenyl]-piperidine-1-carboxylic acid 4-trifluoromethyl-benzyl ester

Example 13-2: (2-Methyl-5-{1-[4-methyl-2-(4-trifluoromethyl-phenyl)-thiazole-5-carbonyl]-piperidin-3-yl}-phenylamino)-acetic acid

### ANNEX B

A preferred aspect of the invention is the use of a PPAR agonist of a formula or structure disclosed below. Methods of making the compounds disclosed in Annex B can be found by reference to the relevant publication, which are all incorporated by reference.

Suitable compounds of the invention include the subject matter of claim 1 of US20040053979A1 and incorporate examples 1-72.

Suitable compounds of the invention include the subject matter of claim 1 of WO2004020420A1 and incorporate examples 1-140.

Suitable compounds of the invention include the subject matter of claim 1 of US2004006116A1 and incorporate examples 1-24.

Suitable compounds of the invention include the subject matter of claim 1 of FR2841900A1 and incorporate examples 26-31.

Suitable compounds of the invention include the subject matter of claim 1 of FR2841784A1 and incorporate examples 29-34.

Suitable compounds of the invention include the subject matter of claim 1 of WO2004000790A1 and incorporate examples 1-15.

Suitable compounds of the invention include the subject matter of claim 1 of WO2004000789A1 and incorporate examples 1-138.

Suitable compounds of the invention include the subject matter of claim 1 of WO2004000295A1 and incorporate examples 1-30.

Suitable compounds of the invention include the subject matter of claim 1 of WO2004000294A1 and incorporate examples 1-14.

Suitable compounds of the invention include the subject matter of claim 1 of WO2003099821A1 and incorporate examples 1-73.

Suitable compounds of the invention include the subject matter of claim 1 of WO2003099793A1 and incorporate examples 1-379.

Suitable compounds of the invention include the subject matter of claim 1 of U.S. Pat. No. 6,653,334B1 and incorporate examples 1-185.

Suitable compounds of the invention include the subject matter of claim 1 of WO2003084916A2 and incorporate examples 1-134.

Suitable compounds of the invention include the subject matter of claim 1 of US2003181494A1 and incorporate examples 1-10.

Suitable compounds of the invention include the subject matter of claim 1 of WO2003074052A1 and incorporate examples 1-7.

Suitable compounds of the invention include the subject matter of claim 1 of WO2003074051A1 and incorporate examples 1-25.

Suitable compounds of the invention include the subject matter of claim 1 of WO2003074050A1 and incorporate examples 1-33.

Suitable compounds of the invention include the subject matter of claim 1 of WO2003072102A1 and incorporate examples 1-133.

Suitable compounds of the invention include the subject matter of claim 1 of WO2003072100A1 and incorporate examples 1-206.

Suitable compounds of the invention include the subject matter of claim 1 of WO2003072099A1 and incorporate examples 1-16.

Suitable compounds of the invention include the subject matter of claim 1 of US2003158232A1 and incorporate examples 1-71.

Suitable compounds of the invention include the subject matter of claim 1 of WO2003059875A2 and incorporate examples 1-26.

Suitable compounds of the invention include the subject matter of claim 1 of WO2003053976A1 and incorporate examples 1-5.

Suitable compounds of the invention include the subject matter of claim 1 of WO2003053974A1 and incorporate examples 1-19.

Suitable compounds of the invention include the subject matter of claim 1 of WO2003051826A1 and incorporate examples 1-3.

Suitable compounds of the invention include the subject matter of claim 1 of WO2003051822A1 and incorporate examples 1-2.

Suitable compounds of the invention include the subject matter of claim 1 of WO2003051821A1 and incorporate examples 1-2.

Suitable compounds of the invention include the subject matter of claim 1 of WO2003048130A2 and incorporate examples 1-221.

Suitable compounds of the invention include the subject matter of claim 1 of WO2003048116A2 and incorporate examples 1-52.

Suitable compounds of the invention include the subject matter of claim 1 of WO2003048108A2 and incorporate examples 1-48.

Suitable compounds of the invention include the subject matter of claim 1 of WO2003043997A1 and incorporate examples 1-15.

Suitable compounds of the invention include the subject matter of claim 1 of WO2003043985A1 and incorporate examples 1-7.

Suitable compounds of the invention include the subject matter of claim 1 of WO2003033481A1 and incorporate examples 1-72.

Suitable compounds of the invention include the subject matter of claim 1 of WO2003020269A1 and incorporate examples 1-46.

Suitable compounds of the invention include the subject matter of claim 1 of WO2003006022A1 and incorporate examples 1-3.

Suitable compounds of the invention include the subject matter of claim 1 of WO2003004458A1 and incorporate examples 1-90.

Suitable compounds of the invention include the subject matter of claim 1 of WO2002100813A2 and incorporate examples 1-379.

Suitable compounds of the invention include the subject matter of claim 1 of WO2002100403A1 and incorporate examples 1-758.

Suitable compounds of the invention include the subject matter of claim 1 of WO2002096895A1 and incorporate examples 1-4 and particularly example 2,2-methyl-2-[4-{[(4-methyl-5-[4-ethylphenyl]thiazol-2-ylcarbonyl)amino]methyl}phenoxy]propionic acid and example 4,2-methyl-2-[4-{[(4-methyl-5-[4-fluorophenyl]thiazol-2-ylcarbonyl)amino]methyl}-phenoxy]propionic acid.

Suitable compounds of the invention include the subject matter of claim 1 of WO2002096894A1 and incorporate examples 1-8, and particularly examples 2 and 4.

Suitable compounds of the invention include the subject matter of claim 1 of WO2002096358A2 and incorporate examples 1-62.

Suitable compounds of the invention include the subject matter of claim 1 of WO2002096357A2 and incorporate examples 1-15.

Suitable compounds of the invention include the subject matter of claim 1 of US2002173663A1 and incorporate examples 1-29.

Suitable compounds of the invention include the subject matter of claim 1 of WO2002092084A1 and incorporate examples 1-8.

Suitable compounds of the invention include the subject matter of claim 1 of WO2002081454A1 and incorporate examples 1-49.

Suitable compounds of the invention include the subject matter of claim 1 of WO2002076177A2 and incorporate examples 1-15.

Suitable compounds of the invention include the subject matter of claim 1 of U.S. Pat. No. 6,444,816B1 and incorporate examples 1-20.

Suitable compounds of the invention include the subject matter of claim 1 of U.S. Pat. No. 6,440,961B1 and incorporate examples 1-34.

Suitable compounds of the invention include the subject matter of claim 1 of WO2002064094A2 and incorporate examples 1-33.

Suitable compounds of the invention include the subject matter of claim 1 of WO2002062798A2 and incorporate examples 1-50.

Suitable compounds of the invention include the subject matter of claim 1 of WO2002062774A1 and incorporate examples 1-57.

Suitable compounds of the invention include the subject matter of claim 1 of US20020103242A1 and incorporate examples 1-29.

Suitable compounds of the invention include the subject matter of claim 1 of WO2002059098A1 and incorporate examples 14-123.

Suitable compounds of the invention include the subject matter of claim 1 of WO2002050047A1 and incorporate examples 1-12.

Suitable compounds of the invention include the subject matter of claim 1 of WO2002046174A1 and incorporate examples 1-20.

Suitable compounds of the invention include the subject matter of claim 1 of WO2002038553A2 and incorporate examples 1-157.

Suitable compounds of the invention include the subject matter of claim 1 of WO2002030914A1 and incorporate examples 1-9.

Suitable compounds of the invention include the subject matter of claim 1 of U.S. Pat. No. 6,369,067B1 and incorporate examples 1-30.

Suitable compounds of the invention include the subject matter of claim 1 of WO2002026729A2 and incorporate examples 1-29.

Suitable compounds of the invention include the subject matter of claim 1 of WO2002018355A1 and incorporate examples 1-63.

Suitable compounds of the invention include the subject matter of claim 1 of WO2002016332A1 and incorporate examples 1-72.

Suitable compounds of the invention include the subject matter of claim 1 of WO2002016331A1 and incorporate examples 1-147.

Suitable compounds of the invention include the subject matter of claim 1 of US2002002200A1 and incorporate examples 1-2.

Suitable compounds of the invention include the subject matter of claim 1 of WO2001087862A2 and incorporate examples 1-132.

Suitable compounds of the invention include the subject matter of claim 1 of WO2001079150A1 and incorporate examples 1-6.

Suitable compounds of the invention include the subject matter of claim 1 of WO2001055086A1 and incorporate examples 1-25.

Suitable compounds of the invention include the subject matter of claim 1 of WO2001055085A1 and incorporate examples 1-155.

Suitable compounds of the invention include the subject matter of claim 1 of WO2001040207A1 and incorporate examples 1-57.

Suitable compounds of the invention include the subject matter of claim 1 of WO2001025226A1 and example 32.

Suitable compounds of the invention include the subject matter of claim 1 of WO2001016120A1 and incorporate examples 1-74.

Suitable compounds of the invention include the subject matter of claim 1 of WO2000078313A1 and incorporate examples 1-12.

Suitable compounds of the invention include the subject matter of claim 1 of WO2000078312A1 and incorporate examples 1-10.

Suitable compounds of the invention include the subject matter of claim 1 of WO2000066572A1 and incorporate examples 1-77.

Suitable compounds of the invention include the subject matter of claim 1 of EP1044966A1 and incorporate examples 1-21.

Suitable compounds of the invention include the subject matter of claim 1 of WO2000023407A2 and incorporate examples 1-5 and in particular example 5,2-(4-(2-(1-heptyl-3-(2,4-difluorophenyl)ureido)ethylphenylthio)-2-methylpropionic acid.

Suitable compounds of the invention include the subject matter of claim 1 of U.S. Pat. No. 6,054,453A and incorporate examples 1-23.

Suitable compounds of the invention include the subject matter of claim 1 of WO2000008002A1 and incorporate examples 1-80.

Suitable compounds of the invention include the subject matter of claim 1 of WO9958510A1 and incorporate examples 1-150.

Suitable compounds of the invention include the subject matter of claim 1 of WO9938850A1 and incorporate examples 1-38.

Suitable compounds of the invention include the subject matter of claim 1 of WO9919313A1 and incorporate examples 1-23.

Suitable compounds of the invention include the subject matter of claim 1 of WO9908501A2 and incorporate examples 1-64.

Suitable compounds of the invention include the subject matter of claim 1 of WO2004005233A1 and incorporate examples 1-42.

Suitable compounds of the invention include the subject matter of claim 1 of WO2004000785A2 and incorporate examples 1-107.

Suitable compounds of the invention include the subject matter of claim 1 of WO2003074495A1 and incorporate examples 1-144.

A suitable compound of the invention includes the compound of claim 1 of WO2002096893A1: 2-methyl-2-[4-{[(4-methyl-2-[4-trifluoromethylphenyl]thiazol-5-yl-carbonyl)amino]methyl}phenoxy]propionic acid.

Suitable compounds of the invention include the subject matter of claim 1 of WO2003011819A1 and incorporate examples 1-81.

Suitable compounds of the invention include the subject matter of claim 1 of WO2002062799A1 and incorporate examples 1-20.

Suitable compounds of the invention include the subject matter of claim 1 of WO2002060434A2 and incorporate examples 1-29.

Suitable compounds of the invention include the subject matter of claim 1 of WO2002060434A2 and incorporate examples 1-29.

Suitable compounds of the invention include the subject matter of claim 1 of US2002055502A1.

Suitable compounds of the invention include the subject matter of claim 1 of WO200226737A1 and incorporate examples 1-5.

Suitable compounds of the invention include the subject matter of claim 1 of WO200160807A1 and incorporate examples 1-29.

Suitable compounds of the invention include the subject matter of claim 1 of WO200063190A1 and incorporate examples 1-2.

Suitable compounds of the invention include the subject matter of claim 1 of WO200063209A1 and incorporate examples 1-4.

Suitable compounds of the invention include the subject matter of claim 1 of WO200064888A1 and incorporate examples 1-59.

Suitable compounds of the invention include the subject matter of claim 1 of U.S. Pat. No. 6,130,214A and incorporate examples 1-34.

Suitable compounds of the invention include the subject matter of claim 1 of FR2781222A1 and incorporate examples 1-81.

Suitable compounds of the invention include the subject matter of claim 1 of WO9932465A1 and incorporate examples 1-75.

Suitable compounds of the invention include the subject matter of claim 1 of WO9916758A1 and incorporate examples 1-34.

Suitable compounds of the invention include the subject matter of claim 1 of WO2004024726A1 and incorporate examples 1-72.

Suitable compounds of the invention include the subject matter of claim 1 of WO2004020408A1 and incorporate examples 1-189.

Suitable compounds of the invention include the subject matter of claim 1 of WO2004005266A1 and incorporate examples 1-26.

Suitable compounds of the invention include the subject matter of claim 1 of WO2004000762A2 and incorporate examples 1-58.

Suitable compounds of the invention include the subject matter of claim 1 of WO2004000315A1 and incorporate examples 1-114.

Suitable compounds of the invention include the subject matter of claim 1 of WO2003066581A1 and incorporate examples 1-68.

Suitable compounds of the invention include the subject matter of claim 1 of WO2003043998A1 and incorporate examples 1-21.

Suitable compounds of the invention include the subject matter of claim 1 of WO2003033453A1 and incorporate examples 1-29.

Suitable compounds of the invention include the subject matter of claim 1 of WO2003018553A1 and incorporate examples 1-127.

Suitable compounds of the invention include the subject matter of claim 1 of US06028088 and incorporate compound 3.

Suitable compounds of the invention include the subject matter of claim 1 of US20040192688A1 and incorporate examples 1-53.

Suitable compounds of the invention include the subject matter of claim 1 of US20040209936A1 and incorporate examples 1-207.

Suitable compounds of the invention include the subject matter of claim 1 of US20040224995A1.

Suitable compounds of the invention include the subject matter of claim 1 of US20040248951A1 and incorporate examples 1-20.

Suitable compounds of the invention include the subject matter of claim 1 of US20040259950A1 and incorporate examples 1-42.

Suitable compounds of the invention include the subject matter of claim 1 of WO00004011A1 and incorporate examples 1-81.

Suitable compounds of the invention include the subject matter of claim 1 of WO00023415A1 and incorporate examples 1-16.

Suitable compounds of the invention include the subject matter of claim 1 of WO00023416A1 and incorporate examples 1-13.

Suitable compounds of the invention include the subject matter of claim 1 of WO00023417A1 and incorporate examples 1-11.

Suitable compounds of the invention include the subject matter of claim 1 of WO00023425A1 and incorporate examples 1-6.

Suitable compounds of the invention include the subject matter of claim 1 of WO00023445A1.

Suitable compounds of the invention include the subject matter of claim 1 of WO00023451A1.

Suitable compounds of the invention include the subject matter of claim 1 of WO00027832A2 and incorporate examples 1-36.

Suitable compounds of the invention include the subject matter of claim 1 of WO00039113A1 and incorporate examples 35-89.

Suitable compounds of the invention include the subject matter of claim 1 of WO00050414A1 and incorporate examples 1-35.

Suitable compounds of the invention include the subject matter of claim 1 of WO00053601A1 and incorporate examples 1-9.

Suitable compounds of the invention include the subject matter of claim 1 of WO00055118A1 and incorporate examples 1-7.

Suitable compounds of the invention include the subject matter of claim 1 of WO00063153A1 and incorporate examples 1-61.

Suitable compounds of the invention include the subject matter of claim 1 of WO00063161A1 and incorporate compounds 1-8.

Suitable compounds of the invention include the subject matter of claim 1 of WO00063190A1.

Suitable compounds of the invention include the subject matter of claim 1 of WO00063196A1 and incorporate examples 1-5.

Suitable compounds of the invention include the subject matter of claim 1 of WO00063209A1 and incorporate examples 1-4.

Suitable compounds of the invention include the subject matter of claim 1 of WO0007831 4A1 and incorporate examples 1-3.

Suitable compounds of the invention include the subject matter of claim 1 of WO02053546A1 and incorporate examples 1-43.

Suitable compounds of the invention include the subject matter of claim 1 of WO02062772A1 and incorporate examples 1-18.

Suitable compounds of the invention include the subject matter of claim 1 of WO02064130A1 and incorporate examples 1-119.

Suitable compounds of the invention include the subject matter of claim 1 of WO02064549A1 and incorporate examples 1-77.

Suitable compounds of the invention include the subject matter of claim 1 of WO2002074291A2.

Suitable compounds of the invention include the subject matter of claim 1 of WO2002080913A1 and incorporate examples 1-77.

Suitable compounds of the invention include the subject matter of claim 1 of WO2002092590A1 and incorporate examples 1-174.

Suitable compounds of the invention include the subject matter of claim 1 of WO02100836A2 and incorporate examples 31-32.

Suitable compounds of the invention include the subject matter of claim 1 of WO03011807A1 and incorporate examples 1-37.

Suitable compounds of the invention include the subject matter of claim 1 of WO03011814A1 and incorporate examples 1-8.

Suitable compounds of the invention include the subject matter of claim 1 of WO03011834A1 and incorporate examples 1-12.

Suitable compounds of the invention include the subject matter of claim 1 of WO03024395A2 and incorporate examples 1-24.

Suitable compounds of the invention include the subject matter of claim 1 of WO03033481A1 and incorporate examples 1-72.

Suitable compounds of the invention include the subject matter of claim 1 of WO03035603A1 and incorporate examples 1-56.

Suitable compounds of the invention include the subject matter of claim 1 of WO03066612A1.

Suitable compounds of the invention include the subject matter of claim 1 of WO03074504A2 and incorporate examples 1-8.

Suitable compounds of the invention include the subject matter of claim 1 of WO03078425A1.

Suitable compounds of the invention include the subject matter of claim 1 of WO03080545A2 and incorporate examples 1-338.

Suitable compounds of the invention include the subject matter of claim 1 of WO03080605A1 and incorporate examples 1-38.

Suitable compounds of the invention include the subject matter of claim 1 of WO03084535A1 and incorporate examples detailed on pages 1-3.

Suitable compounds of the invention include the subject matter of claim 1 of WO03104208A1 and incorporate examples 1-1-4-15.

Suitable compounds of the invention include the subject matter of claim 1 of WO2004005243A2 and incorporate examples 1-42.

Suitable compounds of the invention include the subject matter of claim 1 of WO2004022533A1 and incorporate examples 1-7.

Suitable compounds of the invention include the subject matter of claim 1 of WO2004024939A2 and incorporate examples 1-21.

Suitable compounds of the invention include the subject matter of claim 1 of WO2004031116A1 and incorporate examples 1-14.

Suitable compounds of the invention include the subject matter of claim 1 of WO2004031162A1 and incorporate examples 1-53.

Suitable compounds of the invention include the subject matter of claim 1 of WO2004037213A2 and incorporate examples detailed on pages 52-60.

Suitable compounds of the invention include the subject matter of claim 1 of WO2004037248A2 and incorporate examples 1-91.

Suitable compounds of the invention include the subject matter of claim 1 of WO2004037775A1 and incorporate examples 1-8.

Suitable compounds of the invention include the subject matter of claim 1 of WO2004037776A2 and incorporate examples 1-41.

Suitable compounds of the invention include the subject matter of claim 1 of WO2004037777A1 and incorporate examples 1-60.

Suitable compounds of the invention include the subject matter of claim 1 of WO2004037778A1 and incorporate examples 1-60.

Suitable compounds of the invention include the subject matter of claim 1 of WO2004037779A1 and incorporate examples 1-60.

Suitable compounds of the invention include the subject matter of claim 1 of WO2004037829A1 and incorporate examples 1-6.

Suitable compounds of the invention include the subject matter of claim 1 of WO2004041275A1 and incorporate examples 1-68.

Suitable compounds of the invention include the subject matter of claim 1 of WO2004043951A1 and incorporate examples 1-12.

Suitable compounds of the invention include the subject matter of claim 1 of WO2004046091A2 and incorporate examples 1-37.

Suitable compounds of the invention include the subject matter of claim 1 of WO2004048333A1 and incorporate examples 1-29.

Suitable compounds of the invention include the subject matter of claim 1 of WO2004048334A1 and incorporate examples 1-1-13-2.

Suitable compounds of the invention include the subject matter of claim 1 of WO2004048338A1 and incorporate examples 1-29.

Suitable compounds of the invention include the subject matter of claim 1 of WO2004048351A2 and incorporate examples 1-7.

Suitable compounds of the invention include the subject matter of claim 1 of WO2004052840A1 and incorporate examples 1-22.

Suitable compounds of the invention include the subject matter of claim 1 of WO2004056740A1 and incorporate examples 1-42.

Suitable compounds of the invention include the subject matter of claim 1 of WO2004056748A1 and incorporate examples 1-4.

Suitable compounds of the invention include the subject matter of claim 1 and claim 2 of WO2004058251A1.

Suitable compounds of the invention include the subject matter of claim 1 of WO2004060871A1 and incorporate examples 1-22.

Suitable compounds of the invention include the subject matter of claim 1 of WO2004063148A1 and incorporate examples 1-176.

Suitable compounds of the invention include the subject matter of claim 1 of WO2004063155A1 and incorporate examples 1-110.

Suitable compounds of the invention include the subject matter of claim 1 of WO2004063165A1.

Suitable compounds of the invention include the subject matter of claim 1 of WO2004063184A1 and incorporate examples 1-15.

Suitable compounds of the invention include the subject matter of claim 1 of WO2004063190A1 and incorporate examples 1-73.

Suitable compounds of the invention include the subject matter of claim 1 of WO2004066963A2 and incorporate examples 1-9.

Suitable compounds of the invention include the subject matter of claim 1 of WO2004066964A2 and incorporate examples 1-168.

Suitable compounds of the invention include the subject matter of claim 1 of WO2004071504A1 and incorporate examples 1-141.

Suitable compounds of the invention include the subject matter of claim 1 of WO2004072022A1 and incorporate examples 1-53.

Suitable compounds of the invention include the subject matter of claim 1 of WO2004073606A2 and incorporate examples 1-420.

Suitable compounds of the invention include the subject matter of claim 1 of WO2004074284A1 and incorporate examples 1-43.

Suitable compounds of the invention include the subject matter of claim 1 of WO2004075815A2 and incorporate examples 1-13.

Suitable compounds of the invention include the subject matter of claim 1 of WO2004076401A1 and incorporate examples 1-15.

Suitable compounds of the invention include the subject matter of claim 1 of WO2004076402A1 and incorporate examples 1-62.

Suitable compounds of the invention include the subject matter of claim 1 of WO2004076426A1 and incorporate examples 1-50.

Suitable compounds of the invention include the subject matter of claim 1 of WO2004076427A1 and incorporate examples 1-85.

Suitable compounds of the invention include the subject matter of claim 1 of WO2004076428A1 and incorporate examples 1-91.

Suitable compounds of the invention include the subject matter of claim 1 of WO2004076447A1 and incorporate examples 1-68.

Suitable compounds of the invention include the subject matter of claim 1 of WO2004082621A2 and incorporate examples 1-29.

Suitable compounds of the invention include the subject matter of claim 1 of WO2004092117A1 and incorporate examples 1-41.

Suitable compounds of the invention include the subject matter of claim 1 of WO2004092130A2 and incorporate examples 1-41.

Suitable compounds of the invention include the subject matter of claim 1 of WO2004092131A1 and incorporate examples 1-179.

Suitable compounds of the invention include the subject matter of claim 1 of WO2004092145A1 and incorporate examples detailed on pages 53-162.

Suitable compounds of the invention include the subject matter of claim 1 of WO2004093879A1 and incorporate examples detailed on pages 37-46.

Suitable compounds of the invention include the subject matter of claim 1 of WO2004100945A1 and incorporate examples detailed on pages 5-7.

Suitable compounds of the invention include the subject matter of claim 1 of WO2004103997A1 and incorporate examples 1-14.

Suitable compounds of the invention include the subject matter of claim 1 of WO2004108686A2 and incorporate examples 1-222.

Suitable compounds of the invention include the subject matter of claim 1 of WO2004110983A2 and incorporate examples 1-267.

Suitable compounds of the invention include the subject matter of claim 1 of WO2004110984A1 and incorporate examples 1-11.

Suitable compounds of the invention include the subject matter of claim 1 of WO2004110985A1.

Suitable compounds of the invention include the subject matter of claim 1 of WO2004113270A2 and incorporate examples 1-23.

Suitable compounds of the invention include the subject matter of claim 1 of WO2004113276A1.

Suitable compounds of the invention include the subject matter of claim 1 of WO2004113282A1 and incorporate examples 1-12.

Suitable compounds of the invention include the subject matter of claim 1 of WO2004113283A1.

Suitable compounds of the invention include the subject matter of claim 1 of WO2004113284A1.

Suitable compounds of the invention include the subject matter of claim 1 of WO2004113285A1.

Suitable compounds of the invention include the subject matter of claim 1 of WO2004113331A1 and incorporate examples 1-28.

Suitable compounds of the invention include the subject matter of claim 1 of WO2005000841A1 and incorporate examples 1-53.

Suitable compounds of the invention include the subject matter of claim 1 and claim 2 of WO2005002524A2.

Suitable compounds of the invention include the subject matter of claim 1 of WO2003099766A1 and incorporate examples 1-14.

Suitable compounds of the invention include the subject matter of claim 1 of WO2003091211A1.

Suitable compounds of the invention include the subject matter of claim 1 of JP2003292439A2.

Suitable compounds of the invention include the subject matter of claim 1 of WO2003042194A1.

Suitable compounds of the invention include the subject matter of claim 1 of US2004198774A1 and incorporate example 1.

Suitable compounds of the invention include the subject matter of claim 1 of EP1424330A1 and incorporate examples 1-50.

Suitable compounds of the invention include the subject matter of claim 1 of EP1452521A1 and incorporate examples 1-178.

Suitable compounds of the invention include the subject matter of claim 1 of WO2003014073A1 and incorporate examples 1-5.

Suitable compounds of the invention include the subject matter of claim 1 of WO2003004484A1.

Suitable compounds of the invention include the subject matter of claim 1 of EP1405848A1 and incorporate examples 1-9.

Suitable compounds of the invention include the subject matter of claim 1 of US2004102634A1 and incorporate examples 1-596.

Suitable compounds of the invention include the subject matter of claim 1 of US2004214888A1 and incorporate examples 1-414.

Suitable compounds of the invention include the subject matter of claim 1 of US2004138271A1 and incorporate examples 1-75.

Suitable compounds of the invention include the subject matter of claim 1 of US2004116708A1 and incorporate examples 1-31.

Suitable compounds of the invention include the subject matter of claim 1 of US2004063775A1 and incorporate examples 1-83.

Suitable compounds of the invention include the subject matter of claim 1 of JP2002193948A2.

Suitable compounds of the invention include the subject matter of claim 1 of US2004138213A1 and incorporate examples 1-12.

Suitable compounds of the invention include the subject matter of claim 1 of WO2002046176A1.

Suitable compounds of the invention include the subject matter of claim 1 of EP1348698A1 and incorporate examples 1-28.

Suitable compounds of the invention include the subject matter of claim 1 of WO2002046146A1.

Suitable compounds of the invention include the subject matter of claim 1 of WO2002044131A1.

Suitable compounds of the invention include the subject matter of claim 1 of WO2002044130A1.

Suitable compounds of the invention include the subject matter of claim 1 of WO2002044129A1.

Suitable compounds of the invention include the subject matter of claim 1 of WO2002044127.

Suitable compounds of the invention include the subject matter of claim 1 of JP2002080362A1.

Suitable compounds of the invention include the subject matter of claim 1 of US2003187068A1 and incorporate examples 1-127.

Suitable compounds of the invention include the subject matter of claim 1 of EP1277469A1.

Suitable compounds of the invention include the subject matter of claim 1 of US2003109570A1 and incorporate examples 1-16.

Suitable compounds of the invention include the subject matter of claim 1 of US2003212100A1 and incorporate examples 1-28.

Suitable compounds of the invention include the subject matter of claim 1 of JP2001261612A2.

Suitable compounds of the invention include the subject matter of claim 1 of WO2001038325A1 and incorporate examples 1-346.

Suitable compounds of the invention include the subject matter of claim 1 of U.S. Pat. No. 6,734,199B1 and incorporate examples 1-13.

Suitable compounds of the invention include the subject matter of claim 1 of U.S. Pat. No. 6,545,026B1 and incorporate examples 1-4.

Suitable compounds of the invention include the subject matter of claim 1 of U.S. Pat. No. 6,730,687B1 and incorporate examples 1-27.

Suitable compounds of the invention include the subject matter of claim 1 of U.S. Pat. No. 6,506,797B1 and incorporate examples 1-197.

Suitable compounds of the invention include the subject matter of claim 1 of U.S. Pat. No. 6,821,994B2 and incorporate examples 1-7.

Suitable compounds of the invention include the subject matter of claims 1-10 of U.S. Pat. No. 6,506,757B1.

Suitable compounds of the invention include the subject matter of claim 1 of US2003032671A1 and incorporate examples 1-1-4-3.

Suitable compounds of the invention include the subject matter of claim 1 of WO9911255A1.

Suitable compounds of the invention include the subject matter of claims 1-3 of EP1216980A1 and incorporate examples 1-351.

Suitable compounds of the invention include the subject matter of claim 1 of WO2005005421A1and incorporate examples 1-6 and 7-1-7-15.

Suitable compounds of the invention include the subject matter of claim 1 of US2005004187A1 and incorporate examples 1-35.

Suitable compounds of the invention include the subject matter of claim 1 of WO2004089276A1 and incorporate examples detailed on pages 4-7.

Suitable compounds of the invention include the subject matter of claim 1 of US2004198814A1 and incorporate examples 1-232.

Suitable compounds of the invention include the subject matter of claim 1 of WO2004074239A1 and incorporate examples 1-23.

Suitable compounds of the invention include the subject matter of claim 1 of WO2004063166A1 and incorporate examples 1-163.

Suitable compounds of the invention include the subject matter of claim 1 of US2004122069A1 and incorporate examples 1-68.

Suitable compounds of the invention include the subject matter of claim 1 of WO2004048371A1 and incorporate examples 1-13.

Suitable compounds of the invention include the subject matter of claim 1 of WO2004110419A1.

Suitable compounds of the invention include the subject matter of claim 1 of WO2004103995A1 and incorporate examples 1-35 and particularly preferred examples include example 20, 2-ethylpropane.

Suitable compounds of the invention include the subject matter of claim 1 of WO2002030895A1 and incorporate examples 1-360.

Suitable compounds of the invention include the subject matter of claim 1 of WO2002028821A2 and incorporate examples 1-360.

Suitable compounds of the invention include the subject matter of claim 1 of WO2002050048A1 and incorporate examples 1-12.

Suitable compounds of the invention include the subject matter of claim 1 of WO2002040020A1 and in particular the compound 5-pyridyl)amino)ethoxy]benzyl]thiazolidine-2,4-dione.

Suitable compounds of the invention include the subject matter of claim 6 of WO2002028434A2 and in particular the compound {2-methyl-4-trifluoromethylphenyl)thiazol-5-ylmethylthio]phenoxy}-acetic acid.

Suitable compounds of the invention include the subject matter of claim 6 of WO2002028433A2 and in particular the compound {2-methyl-4-trifluoromethylphenyl)thiazol-5-ylmethylthio]phenoxy}-acetic acid.

Suitable compounds of the invention include the subject matter of claim 1 of WO2001017994A1 and incorporate examples 1-19.

Suitable compounds of the invention include the subject matter of claim 1 of WO2001000603A1 and incorporate examples 1-89.

Suitable compounds of the invention include the subject matter of claim 3 of WO9736579A1.

Suitable compounds of the invention include the subject matter of claim 1 of WO9731907A1 and incorporate examples 1-128.

Suitable compounds of the invention include the subject matter of claim 1 of FR2849849A1 and incorporate examples 1-70.

Suitable compounds of the invention include the subject matter of claim 1 of FR2845087A1 and incorporate examples 1-14.

Suitable compounds of the invention include the subject matter of claim 1 of WO2004020409A1 and incorporate examples 1-24.

Suitable compounds of the invention include the subject matter of claim 1 of WO2004019869A2 and incorporate examples 1-57.

Suitable compounds of the invention include the subject matter of claim 1 of WO2004010992A1 and incorporate examples 1-9.

Suitable compounds of the invention include the subject matter of claim 1 of WO2004010936A1 and incorporate examples 1-24.

Suitable compounds of the invention include the subject matter of claim 1 of WO2004007468A1 and incorporate examples 1-57.

The compound identified within the reference Drug Data Report, 2000,22(10):906, 292538 is a PPARα agonist.

Compounds identified within the reference Drug Data Report, 2001,23(3):266, 296200 and 296201 are PPARα and PPARγ agonists.

Compounds identified within the reference Drug Data Report, 2001,23(3):267, 296212, 296213, 296214, 296216 and 29621 are PPARα and PPARγ agonists.

Compounds identified within the reference Drug Data Report, 2003,25(3):241, 330408, 330413, 330417, 330419 and 330421 are PPARα and PPARγ agonists.

Compounds identified within the reference Drug Data Report, 2003,25(6):523, 337293, 337294, 337295, 337296, 337297, 337298, 337299, 337300, 337301, 337302, 337303 and 337304 are PPARα agonists.

The compound identified within the reference Drug Data Report, 2001,23(8):788, 259635 is a PPARα and a PPARγ agonist.

The compound identified within the reference Drug Data Report, 2001,23(9):890, 275437 is a PPARα and a PPARγ agonist.

The compound identified within the reference Drug Data Report, 2201,23(6):566, 278306 is a PPARα and a PPARγ agonist.

The compound identified within the reference Drug Data Report, 1999,21(11):1034, 280303 is a PPARα agonist.

The compound identified within the reference Drug Data Report, 2000,22(4):338, 285561 is a PPARα and a PPARγ agonist.

Compounds identified within the reference Drug Data Report, 2000,22(5):439, 286461, 286464, 286465, 286466, 286467, 286468 and 286469 are PPARα and PPARγ agonists.

Compounds identified within the reference Drug Data Report, 2000,22(8):709, 289364, 289365, 289366 and 289367 are PPARα and PPARγ modulators.

The compound identified within the reference Drug Data Report, 2003,25(6):572, 292314 is a PPARα and a PPARγ agonist.

Compounds identified within the reference Drug Data Report, 2001,23(2):148, 294059, 294064 and 294065 are PPARα and PPARγ agonists.

The compound identified within the reference Drug Data Report, 2002,24(2):191, 298465 is a PPARα agonist.

Compounds identified within the reference Drug Data Report, 2001,23(8):780, 302204, 302205, 302208, 302209 302210, 302211, 302212, 302213, 302214, 302215 and 302216 are PPARα and PPARγ agonists.

Compounds identified within the reference Drug Data Report, 2001,23(8):781, 302217, 302218, 302219, 302220, 302221, 302222, 302223, 302224 and 302225 are PPARα and PPARγ agonists.

Compounds identified within the reference Drug Data Report, 2001,23(8):782, 302323, 302324, 302325, 302327, 302328 and 302329 are PPARα and PPARγ agonists.

The compound identified within the reference Drug Data Report, 2002,24(5):436, 302324 is a PPARα and a PPARγ agonist.

Compounds identified within the reference Drug Data Report, 2001,23(9):888, 303998 and 303999 are PPARα and PPARγ agonists.

The compound identified within the reference Drug Data Report, 2001,23(9):889, 307374 is a PPARα and a PPARγ agonist.

Compounds identified within the reference Drug Data Report, 2001,23(11):1077, 308993, 308994, 308995, 308996, 308997, 308993, 308999 and 309000 are PPARα and PPARγ agonists.

Compounds identified within the reference Drug Data Report, 2002,24(2):139, 312257, 312259, 312260, 312261, 312262 and 312264 are PPARα and PPARγ agonists.

The compound identified within the reference Drug Data Report, 2002,24(8):759, 322124 is a PPARα agonist.

The compound identified within the reference Drug Data Report, 2002,24(3):240, 314996 is a PPARα and a PPARγ agonist.

The compound identified within the reference Drug Data Report, 2003,25(1):93, 317368 is a PPARα agonist.

Compounds identified within the reference Drug Data Report, 2002,24(6):530, 318903, 318906, 318907, 318909, 318910, 318911, 318912 and 318913 are PPARα and PPARγ agonists.

Compounds identified within the reference Drug Data Report, 2002,24(8):721, 321286, 321287, 321288, 321289, 321290, 321291, 321292, 321293 and 321294 are PPARα and PPARγ agonists.

Compounds identified within the reference Drug Data Report, 2002,24(10):942, 322638, 322639, 322640, 322641 and 322642 are PPARα agonists.

Compounds identified within the reference Drug Data Report, 2002,24(10):943, 322643, 322644, 322645, 322646, 322648 and 322649 are PPARα agonists.

The compound identified within the reference Drug Data Report, 2002,24(9):849, 322744 is a PPARα agonist.

Compounds identified within the reference Drug Data Report, 2003,25(9):806, 324518, 345324, 345325, 345326 and 345327 are PPARα and PPARγ agonists.

Compounds identified within the reference Drug Data Report, 2002,24(11):996, 325428, 325429, 325430, 325431, 325432, 325433, 325434, 325435 and 325437 are PPARα and PPARγ agonists.

Compounds identified within the reference Drug Data Report, 2002,24(11):1045, 325717, 325718, 325719, 325720, 325722, 325724, 325727 and 325728 are PPARα and PPARγ agonists.

Compounds identified within the reference Drug Data Report, 2002,24(11):997, 325956, 325957, 325959, 325960, 325961, 325962, 325963, 325964 and 325966 are PPARα and PPARγ agonists.

Compounds identified within the reference Drug Data Report, 2002,24(11):998, 325970, 325971, 325972, 325973, 325975, 325976, 325978, 325982 and 325984 are PPARα and PPARγ agonists.

Compounds identified within the reference Drug Data Report, 2003,25(3):244, 331410, 331412, 331415, 331416, 331418, 331420, 331423, 331426, 331429, 331433 and 331436 are PPARα and PPARγ agonists.

Compounds identified within the reference Drug Data Report, 2003,25(3):245, 333187 and 333185 are PPARα, PPARγ and PPARδ agonists.

Compounds identified within the reference Drug Data Report, 2003,25(4):344, 333764, 333765, 333766, 333767, 333768, 333769, 333770, 333771 and 333772 are PPARα and PPARγ agonists.

Compounds identified within the reference Drug Data Report, 2003,25(4):345, 334821, 336820 and 336821 are PPARα and PPARγ agonists.

The compounds identified within the reference Drug Data Report, 2003,25(5):433, 335714 is a PPARα and a PPARγ agonist.

Compounds identified within the reference Drug Data Report, 2003,25(7):623, 339425, 339426, 339427 and 339428 are PPARα and PPARγ agonists.

Compounds identified within the reference Drug Data Report, 2003,25(9):809, 347878, 347879, 347880, 347881, 347882, 347884, 347885, 347886 and 347887 are PPARα and PPARγ agonists.

Compounds identified within the reference Drug Data Report, 2003,25(11):995, 354383, 354384, 354385, 354386, 354387, 354388 and 354389 are PPARα and PPARγ agonists.

The compound identified within the reference Drug Data Report, 2004,26(2):141, 359331 is a PPARα and PPARγ agonist.

Compounds identified within the reference Drug Data Report, 2004,26(2):141, 359334 and 359335 are PPARα agonists.

Compounds identified within the reference Drug Data Report, 2004,26(2):142, 359336, 359337, 359338 and 359339 are PPARα agonists.

The compound identified within the reference Bioorg. Med. Chem. Lett., 13, No 5, 931-35, 2003: Example 9, is a PPARα and a PPARγ agonist.

The compound identified within the reference Bioorg. Med. Chem. Lett., 13, No 16, 2795-98, 2003: Example 12, is a PPARα and a PPARγ agonist.

The compound identified within the reference Bio Bioorg. Med. Chem. Lett., 13, No 19, 3185-90, 2003: Example 5, is a PPARα and a PPARγ agonist.

The compound identified within the reference Bio Bioorg. Med. Chem. Lett., 13, No 3, 399-403, 2003: Example 2c, is a PPARα and a PPARγ agonist.

The compound identified within the reference Bio Bioorg. Med. Chem. Lett., 13, No 20, 3541-44, 2003: Example 12, is a PPARα and a PPARγ agonist.

The compound identified within the reference Bio Bioorg. Med. Chem. Lett., 12, No 3, 333-35, 2002: Example (+)-5, is a PPARα and a PPARγ agonist.

The compound identified within the reference Bio Bioorg. Med. Chem. Lett., 42, No 19, 3785-88, 1999: GW-9578, is a PPARα and a PPARγ agonist.

The compound identified within the reference J. Med. Chem., 46, No 17, 3581-99, 2003: Compound 10, is a PPARα agonist.

The compound identified within the reference J. Pharmacology & Experimental Therapeutics, 309, No 3, 970-977, 2004: NS-220 is a PPARα agonist.

Suitable compounds of the invention also include compounds identified within the reference Winegar D A, Role of peroxisome proliferator-activated receptors in atherosclerosis, Current Opinion in Cardiovascular, Pulmonary & Renal Investigational Drugs, 2000, Vol 2 No 3: pages 235, in table 1, in particular PPARα agonists: Beclofibrate, Bezafibrate, Ciprofibrate, Clofibrate, Etofylline-clofibrate, Fenofibrate, Gemfibrozil, GW-9820, WY-14646 and GW-9578; and PPARγ agonists: Troglitazone, Pioglitazone, Rosiglitazone, GI-262570, Darglitazone, Isaglitazone, Englitazone, GW-7845, LY-300512, GW-1929, AD-5075 and L-796449.

Suitable compounds of the invention also include compounds identified within reference: Wilson T, Brown P, Sternbach D, Henke B: The PPARs: from orphan receptors to drug discovery. Journal Med Chem (2000) 43: 527-550.

Suitable compounds of the invention also include compounds identified within reference: Hertz R, Bishara-Shieban J, Bar-Tana J: Mode of action of peroxisome proliferators as hypolipidemic drugs.

Suppression of apolipoprotein C-III. Journal Biol Chem (1995) 270: 13470-13475.

Suitable compounds of the invention also include compounds identified within reference: Gaw A, Shepherd J: Fibric acid derivatives. Curr Opin Lipidol (1991) 2:39-42.

Suitable compounds of the invention also include compounds identified within reference: Brown P, Winegar D, Plunket K, Moore L, Lewis M, Wilson J, Sundseth S, Koble C, Wu Z, Chapman J, Lehmann J, Kliewer S, Wilson T: A ureido-thioisobutyric acid (GW9578) is a subtype-sensitive PPARα agonist with potent lipid-lowering activity, Journal Med Chem (1999) 42: 3785-3788.

Suitable compounds of the invention also include compounds identified within reference: Wilson T, Cobb J, Cowan D, Wiethe R. Correa I, Prakash S, Beck K, Moore L, Kliewer S, Lehmann J: The structure-activity relationship between peroxisome proliferators-activated receptor γ agonism and the anti-hyperglycemic activity of thiazolidinediones. Journal Med Chem (1996) 39:665-668.

Suitable compounds of the invention also include compounds identified within reference: Berger J, Leibowitz M, Doebber T, Elbrecht A, Zhang B, Zhou G, Biswas C, Cullinan C, Hayes N, Li Y, Tanen M, Ventre J, Wu M, Berger R, Mosley R, Marquis R, Santini C, Sahoo S, Tolman R, Smith R, Moller D: Novel peroxisome proliferator-activated receptor (PPAR) γ and PPARδ ligands produce distinct biological effects. Journal Biol Chem (1999) 274:6718-6275.

Suitable compounds of the invention include the subject matter of claim 1 of WO2004024705A1.

Suitable compounds of the invention include the subject matter of claim 1 of WO2004019927A1.

Suitable compounds of the invention include the subject matter of claim 1 of US2003191144A1 and incorporate examples 1-5.

Suitable compounds of the invention include the subject matter of claim 1 of US2003181434A1.

Suitable compounds of the invention include the subject matter of claim 1 of US2003134885A1 and incorporate examples 1-34.

Suitable compounds of the invention include the subject matter of claim 1 of FR2833949A1 and incorporate examples 1-23.

Suitable compounds of the invention include the subject matter of claim 1 of US2003109560A1 and incorporate examples 1-8.

Suitable compounds of the invention include the subject matter of claim 1 of JP2003128639A1.

Suitable compounds of the invention include the subject matter of claim 1 of JP2003128539A1.

Suitable compounds of the invention include the subject matter of claim 1 of US2003083329A1 and incorporate compounds 1-132.

Suitable compounds of the invention include the subject matter of claim 1 of WO2003016265A1.

Suitable compounds of the invention include the subject matter of claim 1 of WO2002102780A1.

Suitable compounds of the invention include the subject matter of claim 1 of WO20021 00812A1.

Suitable compounds of the invention include the subject matter of claims 1 and 2 of WO2002100413A1.

Suitable compounds of the invention include the subject matter of claim 1 of WO2002098840A1.

Suitable compounds of the invention include the subject matter of claim 1 of WO2002096880A1.

Suitable compounds of the invention include the subject matter of claim 1 of JP2002338555A1.

Suitable compounds of the invention include the subject matter of claim 1 of WO2002080899A1.

Suitable compounds of the invention include the subject matter of claim 1 of WO2002079162A1.

Suitable compounds of the invention include the subject matter of claim 1 of WO2002076957A1 and particularly [4-[4-[2-[2-chlorophenyl)-5-isopropyloxazol-4-yl]butyryl]phenyl]acetonitrile.

Suitable compounds of the invention include the subject matter of claim 1 of WO2002071827A2 and incorporate examples 1-65.

Suitable compounds of the invention include the subject matter of claim 1 of WO2002051820A1.

Suitable compounds of the invention include the subject matter of claim 1 of JP2002179568A1.

Suitable compounds of the invention include the subject matter of claim 1 of U.S. Pat. No. 6,353,011B1 and incorporate examples 1-9.

Suitable compounds of the invention include the subject matter of claim 1 of WO2002013864A1.

Suitable compounds of the invention include the subject matter of claim 1 of WO2002012210A1 and incorporate examples 1-86.

Suitable compounds of the invention include the subject matter of claim 1 of WO2002008188A1 and incorporate examples 1-31.

Suitable compounds of the invention include the subject matter of claim 1 of WO2002000633A1 and incorporate compounds 1-505.

Suitable compounds of the invention include the subject matter of claim 1 of WO2001087860A2 and incorporate examples 1-2.

Suitable compounds of the invention include the subject matter of claim 1 of WO2001083427A1 and in particular, N-[4-(4-methylpiperazin-1-ylcarbonyl)phenyl]-(2-chloro-5-nitrophenyl)carboxamide.

Suitable compounds of the invention include the subject matter of claim 1 of WO2001080854A1.

Suitable compounds of the invention include the subject matter of claim 1 of JP2001131173A1.

Suitable compounds of the invention include the subject matter of claim 1 of JP2001097954A1.

Suitable compounds of the invention include the subject matter of claim 1 of U.S. Pat. No. 6,706,763B1 and incorporate examples 1-9.

Suitable compounds of the invention include the subject matter of claim 1 of U.S. Pat. No. 6,780,431B1 and incorporate examples 1-9 and particularly example 1, N-[(4-nitrophenyl)methyl]-5-[(2,4-dioxothiazolidin-5-yl)methyl]-2-methoxybenzamide.

Suitable compounds of the invention include the subject matter of claim 1 of U.S. Pat. No. 6,787,556B1 and incorporate examples 1-12.

Suitable compounds of the invention include the subject matter of claim 1 of WO2001012187A2 and incorporate examples 1-31.

Suitable compounds of the invention include the subject matter of claim 1 of WO2001000579A1 and incorporate examples 1-372.

Suitable compounds of the invention include the subject matter of claim 1 of WO2000064876A1 and incorporate examples 1-104.

Suitable compounds of the invention include the subject matter of claim 1 of WO2000063161A1 and incorporate examples 1-8.

Suitable compounds of the invention include the subject matter of claim 1 of WO9938845A1 and incorporate examples 1-49.

Suitable compounds of the invention include the subject matter of claim 1 of WO9915520A1.

Suitable compounds of the invention include the subject matter of claim 1 of WO2004110982A1.

Suitable compounds of the invention include the subject matter of claim 1 of WO2004069241A1 and incorporate examples 1-6.

Suitable compounds of the invention include the subject matter of claim 1 of WO2004073593A2 and incorporate examples 1-23.

Suitable compounds of the invention include the subject matter of claim 1 of WO2004073698A1 and incorporate examples 1-26.

Annex C

A preferred aspect of the invention is the use of a PPAR agonist of a formula or structure disclosed below. Methods of making the compounds disclosed in Annex C can be found by reference to the relevant publication, which are all incorporated by reference.

Suitable compounds of the invention include the subject matter of claim 1 of PCT/IB02/00043, WO2002064130 and includes Examples 1-119.

Suitable compounds of the invention include the subject matter of claim 1 of PCT/IB02/00045, WO2002064549, and includes Examples 1-77

Suitable compounds of the invention include the subject matter of claim 1 of PCT/IB02/02843, WO 03/0185538, and includes Examples 1-127

Suitable compounds of the invention include the subject matter of claim 1 of PCT/IB04/001159, WO 2004/092145, and includes Examples A1-A22, B1-B29, C1-C95 and D1-D43.

Suitable compounds of the invention include the subject matter of claim 1 of PCT/IB03/01131, WO 03/082276, and includes Examples 1-1 to 1-15.

Suitable compounds of the invention include the subject matter of claim 1 of PCT/IB2004/000338, WO2004-074284, and includes Examples 1-43

Suitable compounds of the invention include the subject matter of claim 1 of PCT/IB03/00817, WO 2003/074052, and includes Examples 1-7

Suitable compounds of the invention include the subject matter of claim 1 of PCT/IB03/00882, WO 03/074051, and includes Examples 1-25

Suitable compounds of the invention include the subject matter of claim 1 of PCT/IB04/001178, WO 04/091604, and includes Examples 1-207

1. The use of a PPAR agonist to increase ruminant serum glucose concentration.

2. The use according to claim 1, with the proviso that a compound of formula I, Annex A, is not used.

3. The use according to claim 1, wherein the PPAR agonist is a PPAR alpha selective agonist.

4. The use as claimed in claim 1, wherein the PPAR agonist is of a formula selected from the formulae disclosed in Annex A, Annex B or Annex C.

5. The use as claimed in claim 4, wherein the PPAR agonist is of a formula disclosed in Annex C.

6. The use as claimed in claim 5, wherein the PPAR agonist is a compound of formula (I) of WO2003084916A2.

7. The use according to claim 1 for the palliative, prophylactic or curative treatment of ruminant diseases associated with reduced serum glucose concentration.

8. The use according to claim 1 for the palliative, prophylactic or curative treatment of ruminant diseases wherein the ruminant disease associated with reduced serum glucose concentration is selected from fatty liver syndrome, dystocia, immune dysfunction, impaired immune function, toxification, primary ketosis, secondary ketosis, downer cow syndrome, indigestion, inappetence, retained placenta, displaced abomasum, mastitis, (endo-)-metritis, infertility, low fertility, lameness, subacute rumen acidosis and inadequate nutrient intake associated with stress e.g. heat, poor housing, overcrowding, shipping, dominance or illness.

9. The use claim 1 wherein the excessive accumulation of triglycerides in liver tissue is prevented or alleviated.

10. The use according to claim 1 for the palliative, prophylactic or curative treatment of fatty liver.

11. The use according to claim 1 wherein the excessive elevation of non-esterified fatty acid levels in serum is prevented or alleviated.

12. The use according to any one of claim 1 wherein the PPAR agonist is administered during the period from 30 days prepartum to 70 days postpartum.

13. The use according to claim 1 wherein the PPAR agonist is administered up to three times during the first seven days postpartum.

14. The use according claim 1 wherein the PPAR agonist is administered at parturition.

15. The use as claimed in claim 1 wherein ruminant milk quality and/or milk yield is increased.

16. The use according to claim 1 wherein peak milk yield is increased.

17. The use according to claim 1 wherein an overall increase in ruminant milk yield is obtained during the 305 days of the bovine lactation period.

18. The use according to claim 1 wherein an overall increase in ruminant milk yield is obtained during the first 60 days of the bovine lactation period.

19. The use according to claim 1 wherein the PPAR agonist is administered to a healthy ruminant.
